SEQUENCE LISTING (10 sequences)

SEQ ID NO: 1 - protein, 233 aa, Staphylococcus sp
SEKSEEINEKDLRKKSELQRNALSNLRQIYYYNEKAITENKESDDQFLENTLLFKGFFTGHPWYNDLLVDLGSKDATNKYKGKKVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNKTINSENLHIDLYLYTT

SEQ ID NO: 2 - protein, 233 aa, Staphylococcus sp
SEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTS

SEQ ID NO: 3 - protein, 233 aa, Artificial Sequence
  Mutated Protein
SEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 4 - protein, 233 aa, Artificial Sequence
  Mutated Protein
SEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIAIYLYTS

SEQ ID NO: 5 - protein, 679 aa, Artificial Sequence
  Conjugated Protein
  MISC_FEATURE (460)..(679): Light Chain
QVQLQQPGAELVRPGASVKLSCKASGYTFTNYWINWVKQRPGQGLEWIGNIYPSYIYTNYNQEFKDKVTLTVDESSSTAYMQLSSPTSEDSAVYYCTRSPYGYDEYGLDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTSDIVMTQSPSSLTVTAGEKVTMNCKSSQSLLNSRNQKNYLTWYQQKPGQPPKLLIYWASTRESGVPDRFTGSGSGTDFTLTISSVQAEDLAVYYCQNDYVYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 6 - protein, 672 aa, Artificial Sequence
  Mutated and Conjugated Protein
  MISC_FEATURE (459)..(672): Light Chain
EVQLQQSGPDLVKPGASVKISCKASGYSFTGYYMHWVKQSHGKSLEWIGRINPNNGVTLYNQKFKDKAILTVDKSSTTAYMELRSLTSEDSAVYYCARSTMITNYVMDYWGQVTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIAIYLYTSSIVMTQTPTSLLVSAGDRVTITCKASQSVSNDVAWYQQKPGQSPKLLISYTSSRYAGVPDRFSGSGSGTDFTLTISSVQAEDLAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 7 - protein, 672 aa, Artificial Sequence
  Mutated and Conjugated Protein
  MISC_FEATURE (459)..(672): Light Chain
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTTSIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 8 - protein, 458 aa, Artificial Sequence
  Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 9 - protein, 214 aa, Artificial Sequence
  Synthetic polypeptide
SIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 10 - protein, 233 aa, Artificial Sequence
  Mutated Protein
SEKSEEINEKDLRKKSELQGTALGNLKQIYYYNEKAITENKESDDQFLENTLLFKGFFTGHPWYNDLLVDLGSKDATNKYKGKKVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNKTINSENLHIALYLYTT

(19) United States
(12) Patent Application Publication
(10) Pub. No.: US 2022/0213194 A1
(43) Pub. Date: Jul. 7, 2022

(54) CANCER TREATMENT

(71) Applicant: NeoTX Therapeutics Ltd., Rehovot (IL)

(72) Inventors: Michal Shahar, Rishon Lezion (IL); Meir Azulay, Givatayim (IL); Asher Nathan, Rishon LeZion (IL)

(21) Appl. No.: 17/610,897

(22) PCT Filed: May 15, 2020
(86) PCT No.: PCT/IL2020/050533

§ 371 Date: Nov. 12, 2021

Related U.S. Application Data
(60) Provisional application No. 62/848,518, filed on May 15, 2019.

(51) Int. Cl. C07K 16/28; C07K 16/30; A61K 47/68; A61P 35/00

(52) U.S. Cl. CPC C07K 16/2818; C07K 16/30; A61P 35/00; A61K 47/6851; A61K 47/6829

(57) ABSTRACT

The invention provides methods or compositions for treating cancer using a superantigen conjugate optionally in combination with a PD-1-based inhibitor.

### CROSS REFERENCE TO RELATED APPLICATIONS

This application claims the benefit of, and priority to, U.S. Provisional Patent Application Ser. No. 62/848,518, filed May 15, 2019, the entire contents of which are hereby incorporated by reference in their entirety.

### FIELD OF THE INVENTION

The invention relates generally to methods and compositions for treating cancer using a superantigen conjugate optionally in combination with an immunopotentiator, e.g., a PD-1-based inhibitor.

### BACKGROUND

According to the American Cancer Society, more than one million people in the United States are diagnosed with cancer each year. Cancer is a disease that results from uncontrolled proliferation of cells that were once subject to natural control mechanisms but have been transformed into cancerous cells that continue to proliferate in an uncontrolled manner. In recent years, a number of immunotherapies have been developed that have attempted to harness the subject's immune system to find and destroy cancer cells. Such immunotherapies include, for example, those that are designed to boost the body's natural defenses for fighting cancer using natural molecules made by the body, or alternatively, through administration of recombinant molecules designed to improve, better target or restore immune system function. Certain immunotherapies include the administration of compounds known to be general immune system enhancers, such as cytokines, for example, IL-2 and interferon. While various immunotherapies developed to date have shown efficacy, they can be associated with side effects including, for example, off-target activities, allergic reactions to the active agents administered including the potential for cytokine storms, a loss of potency caused by the stimulation of antibodies that bind and neutralize the active agents, a decrease in blood cell number, and fatigue.

Other immunotherapies utilize molecules referred to as immune checkpoint inhibitors, which enhance immune responses to cancer. Such checkpoint inhibitors function to inhibit the ability of cancer cells to block immune inhibitory checkpoints thereby resulting in an enhancement of potency of an anti-cancer therapy. A first-generation immune checkpoint inhibitor ipilimumab (YERVOY®; Bristol-Myers Squibb) was approved by the U.S. Food and Drug Administration in 2011 and is an IgG1 monoclonal antibody that can cause ADCC-mediated regulatory T-cell (Treg) cytotoxicity. Over the years, immunochemotherapy, the combination of immunotherapy and chemotherapy, has become important in the treatment of certain cancers. For example, rituximab (RITUXAN®; Roche) is a CD-20-specific monoclonal antibody that depletes CD20-expressing cells and has become a standard component of the treatment of B-cell lymphomas, for example, non-Hodgkin's lymphoma using rituximab (R), cyclophosphamide (C), hydroxydaunorubicin (H), oncovin (O), and prednisone (P), known as R-CHOP.

Recently, PD-1 inhibitors have been approved such as nivolumab and pembrolizumab, which prevent the inhibitory signals between PD-1 and PD-L1. While these drugs have potentiated durable responses in some patients, the response rates of these drugs as monotherapy have been low and in the range of 21%, and the complete response rate has been about 1% in several studies.

Although there are ongoing efforts to combine various cancer therapies to improve patient outcomes and some combinations have shown benefits in efficacy, safety has become a major concern as combining drugs may potentiate serious side effects. For example, drug-related adverse events of grade 3 or 4 were reported in a significant number of patients who received anti-CTLA-4 and anti-PD-1 antibodies in combination as compared to patients who received the anti-CTLA-4 antibody alone.

Accordingly, despite the significant developments that have been made in the fields of immunotherapy and oncology, there is still a need for safe and effective immunotherapies for treating cancer.

### SUMMARY OF THE INVENTION

In recent years, a number of immunotherapies have been developed that have attempted to harness the subject's immune system to find and destroy cancer cells. Although a human immune system has the potential to eliminate cancer cells, certain cancer cells develop the ability to “turn off,” “down regulate,” or otherwise evade the host's immune system allowing the cancerous cells to continue to grow and proliferate unchecked.

The invention is based, in part, upon the discovery that a targeted immune response against a cancer in a subject can be significantly enhanced by combining a superantigen conjugate comprising a superantigen covalently linked to a targeting moiety that binds a cancer antigen (e.g., 5T4) with an immunopotentiator, e.g., a PD-1-based inhibitor.

Furthermore, it has been discovered that administration of a superantigen conjugate comprising a superantigen covalently linked to a targeting moiety that binds a cancer antigen (e.g., 5T4) can promote anticancer immune memory. Without wishing to be bound by theory, it is contemplated that the superantigen conjugate may promote epitope spreading, i.e., the superantigen conjugate can elicit an initial immune response in the subject mediated by the superantigen (which is directed to an epitope on the cancer antigen through the superantigen conjugate) which then spreads to one or more immune responses against other epitopes on other cancer antigens. As a result, the superantigen conjugate, optionally in combination with an immunopotentiator, e.g., a PD-1-based inhibitor, can treat a cancer, treat an otherwise refractory cancer, delay recurrence of a cancer, and/or reduce the likelihood of recurrence of a cancer.

In one aspect, the invention provides a method of reducing the likelihood of a recurrence of a cancer in a subject in need thereof. The method comprises administering to the subject: (i) an effective amount of a superantigen conjugate comprising a superantigen covalently linked to a targeting moiety that binds a cancer antigen (e.g., 5T4 or EpCAM) expressed by cancerous cells within the subject; and optionally (ii) an effective amount of an immunopotentiator, e.g., a PD-1 or PD-L1 inhibitor.

In another aspect, the invention provides a method of delaying a recurrence of a cancer in a subject in need thereof. The method comprises administering to the subject: (i) an effective amount of a superantigen conjugate comprising a superantigen covalently linked to a targeting moiety that binds a cancer antigen (e.g., 5T4 or EpCAM) expressed by cancerous cells within the subject; and optionally (ii) an effective amount of an immunopotentiator, e.g., a PD-1 or PD-L1 inhibitor.

In another aspect, the invention provides a method of treating cancer and promoting anticancer immune memory and/or epitope spreading in a subject in need thereof. The method comprises administering to the subject: (i) an effective amount of a superantigen conjugate comprising a superantigen covalently linked to a targeting moiety that binds a cancer antigen (e.g., 5T4 or EpCAM) expressed by cancerous cells within the subject; and optionally (ii) an effective amount of an immunopotentiator, e.g., a PD-1 or PD-L1 inhibitor.

In another aspect, the invention provides a method of promoting anticancer immune memory and/or epitope spreading in a subject with cancer. The method comprises administering to the subject: (i) an effective amount of a superantigen conjugate comprising a superantigen covalently linked to a targeting moiety that binds a cancer antigen (e.g., 5T4 or EpCAM) expressed by cancerous cells within the subject; and optionally (ii) an effective amount of an immunopotentiator, e.g., a PD-1 or PD-L1 inhibitor.

In another aspect, the invention provides a method of inducing at least a first and second epitope-specific immune response in a subject with cancer. The method comprises administering to the subject: (i) an effective amount of a superantigen conjugate comprising a superantigen covalently linked to a targeting moiety that binds a cancer antigen (e.g., 5T4 or EpCAM) expressed by cancerous cells within the subject; and optionally (ii) an effective amount of an immunopotentiator, e.g., a PD-1 or PD-L1 inhibitor, wherein the first epitope-specific immune response is directed to the cancer antigen through the superantigen conjugate and the second epitope-specific immune response is not directed to the cancer antigen or the superantigen and is mediated by epitope spreading.

In another aspect, the invention provides a method of mediating a long term (for example, at least 6 months, 7 months, 8 month, 9 months, 10 months, 11 months, 1 year, 2 years, 3 years, 4 years, 5 years, 6 years, 7 years, 8 years, 9 years, 10 years, or more) immune response against multiple, different cancer antigens expressed by a cancerous cell in a subject in need of treatment. The method comprises (or consists essentially of) administering to the subject: (i) an effective amount of a superantigen conjugate comprising a superantigen covalently linked to a targeting moiety that binds a single type of cancer antigen expressed by cancerous cells within the subject; and optionally (ii) an effective amount of an immunopotentiator.

In certain embodiments of any of the foregoing methods, the cancer is a 5T4-expressing cancer. In certain embodiments, the cancer is selected from breast cancer, bladder cancer, cervical cancer, colon cancer, colorectal cancer, endometrial cancer, gastric cancer, head and neck cancer, liver cancer, melanoma, mesothelioma, non-small cell lung cancer, ovarian cancer, pancreatic cancer, prostate cancer, renal cell cancer, and skin cancer. In certain embodiments, the cancer is selected from colon cancer and colorectal cancer.

In another aspect, the invention provides a method of stimulating an immune response in a subject against a cancerous cell which does not express a 5T4 cancer antigen. The method comprises administering to the subject: (i) an effective amount of a superantigen conjugate comprising a superantigen covalently linked to a targeting moiety that binds a 5T4 cancer antigen expressed by cancerous cells within the subject; and optionally (ii) an effective amount of an immunopotentiator, e.g., a PD-1 or PD-L1 inhibitor. In another aspect, the invention provides a method of stimulating an immune response in a subject against a cancerous cell which does not express an EpCAM cancer antigen. The method comprises administering to the subject: (i) an effective amount of a superantigen conjugate comprising a superantigen covalently linked to a targeting moiety that binds an EpCAM cancer antigen expressed by cancerous cells within the subject; and optionally (ii) an effective amount of an immunopotentiator, e.g., a PD-1 or PD-L1 inhibitor. In certain embodiments, the cancerous cell is selected from a breast cancer, bladder cancer, cervical cancer, colon cancer, colorectal cancer, endometrial cancer, gastric cancer, head and neck cancer, liver cancer, melanoma, mesothelioma, non-small cell lung cancer, ovarian cancer, pancreatic cancer, prostate cancer, renal cell cancer, and skin cancer cell. In certain embodiments, the cancerous cell is selected from a colon cancer and colorectal cancer cell.

In another aspect, the invention provides a method of treating colon or colorectal cancer in a subject in need thereof. The method comprises administering to the subject: (i) an effective amount of a superantigen conjugate comprising a superantigen covalently linked to a targeting moiety that binds a cancer antigen (e.g., 5T4 or EpCAM) expressed by cancerous cells within the subject; and optionally (ii) an effective amount of an immunopotentiator, e.g., a PD-1 or PD-L1 inhibitor.

In certain embodiments of the any of the foregoing methods, the cancer antigen is selected from EpCAM and 5T4. In certain embodiments, the cancer antigen is 5T4.

In certain embodiments of the any of the foregoing methods, the subject has previously received a different anti-cancer therapy, e.g., an anti-cancer therapy comprising administration to the subject of a chimeric antigen receptor (CAR) T-cell or a bispecific T-cell engager (BiTE). In certain embodiments, the cancer is refractory to the anti-cancer therapy and/or the cancer recurred following the anti-cancer therapy.

In certain embodiments of the any of the foregoing methods, the superantigen conjugate is administered to the subject before, at the same time as, or after the immunopotentiator.

In certain embodiments of the any of the foregoing methods, the superantigen comprises Staphylococcal enterotoxin A or an immunologically variant and/or fragment thereof. In certain embodiments, the superantigen comprises the amino acid sequence of SEQ ID NO: 3, or an immunologically reactive variant and/or fragment thereof.

In certain embodiments of the any of the foregoing methods, the targeting moiety is an antibody, e.g., an anti-5T4 antibody, e.g., an anti-5T4 antibody comprising a Fab fragment that binds a 5T4 cancer antigen. In certain embodiments, the anti-5T4 antibody comprises a heavy chain comprising amino acid residues 1-222 of SEQ ID NO: 8 and a light chain comprising amino acid residues 1-214 of SEQ ID NO: 9. In certain embodiments, the superantigen conjugate comprises a first protein chain comprising SEQ ID NO: 8 and a second protein chain comprising SEQ ID NO: 9.

In certain embodiments of the any of the foregoing methods, the PD-1 inhibitor is an anti-PD-1 antibody, e.g., an anti-PD-1 antibody selected from nivolumab pembrolizumab, and cemiplimab. In certain embodiments, the PD-L1 inhibitor is an anti-PD-L1 antibody, e.g., an anti-PD-L1 antibody selected from atezolizumab, avelumab, and durvalumab.

These and other aspects and features of the invention are described in the following detailed description, figures, and claims.

### BRIEF DESCRIPTION OF THE DRAWINGS

The foregoing and other objects, features and advantages of the invention will become apparent from the following description of preferred embodiments, as illustrated in the accompanying drawings. Like referenced elements identify common features in the corresponding drawings. The drawings are not necessarily to scale, with emphasis instead being placed on illustrating the principles of the present invention.

FIG. 1 is a schematic representation of an exemplary treatment method of the invention using a superantigen conjugate and a PD-1-based inhibitor.

FIG. 2 is a sequence alignment showing the homologous A-E regions in certain wild type and modified superantigens.

FIG. 3 is an amino acid sequence corresponding to an exemplary superantigen conjugate, naptumomab estafenatox/ANYARA®, which comprises two protein chains. The first protein chain comprises residues 1 to 458 of SEQ ID NO: 7 (see also, SEQ ID NO: 8), and includes a chimeric 5T4 Fab heavy chain, corresponding to residues 1 to 222 of SEQ ID NO: 7, and the SEA/E-120 superantigen, corresponding to residues 226 to 458 of SEQ ID NO: 7, covalently linked via a GGP tripeptide linker, corresponding to residues 223-225 of SEQ ID NO: 7. The second chain comprises residues 459 to 672 of SEQ ID NO: 7 (see also, SEQ ID NO: 9) and includes a chimeric 5T4 Fab light chain. The two protein chains are held together by non-covalent interactions between the Fab heavy and light chains.

FIGS. 4A-4D are line graphs illustrating the effect of C215Fab-SEA and anti-PD-1 mAb alone or in combination on tumor volume in the MC38-EpCAM mouse colon tumor model. Tumor growth for individual mice in each treatment group (n=10) is depicted, with FIG. 4A representing vehicle control, FIG. 4B representing anti-PD-1 mAb alone, FIG. 4C representing C215Fab-SEA alone, and FIG. 4D representing C215Fab-SEA and anti-PD-1 mAb in combination. TTS=C215Fab-SEA; aPD1=anti-PD-1 mAb; and TF=tumor free (i.e., complete response).

FIG. 5 is a line graph illustrating the effect of C215Fab-SEA and anti-PD-1 mAb alone or in combination on tumor volume in the MC38-EpCAM mouse colon tumor model. Data depicted are the mean and SE of the individual tumor volumes shown in FIG. 4. ***p<0.0001 at day 19 treatment vs. control; and at day 22 combination vs. TTS or anti-PD-1 alone. TTS=C215Fab-SEA; aPD1=anti-PD-1 mAb; and TGI=tumor growth inhibition.

FIG. 6 is a line graph illustrating long term survival in the MC38-EpCAM mouse colon tumor model after three cycles of treatment with C215Fab-SEA and anti-PD-1 mAb alone or in combination. n=10 for each treatment group. *p=0.02; **p=0.006; ***p=0.0002; TTS=C215Fab-SEA; aPD1=anti-PD-1 mAb; and CR=complete response.

FIG. 7 is a line graph illustrating body weight in the MC38-EpCAM mouse colon tumor model after treatment with C215Fab-SEA and anti-PD-1 mAb alone or in combination. n=10 mice for each treatment group (initially, with the * indicating the number of mice remaining at day 32 for each treatment group). TTS=C215Fab-SEA; and aPD1=anti-PD-1 mAb.

FIG. 8 is a line graph illustrating tumor volume following rechallenge of mice that were previously treated with and completely responded to C215Fab-SEA or C215Fab-SEA and anti-PD-1 mAb therapy with MC38-EpCAM colon cancer cells. TTS=C215Fab-SEA; αPD1=anti-PD-1 mAb; and TV=tumor volume.

FIG. 9 is a line graph illustrating tumor volume following rechallenge of mice that were previously treated with and completely responded to C215Fab-SEA or C215Fab-SEA and anti-PD-1 mAb therapy with parental MC38 colon cancer cells. TTS=C215Fab-SEA; and aPD1=anti-PD-1 mAb; and TV=tumor volume.

FIGS. 10A and 10B are line graphs illustrating percent survival of mice that were previously treated with and completely responded to C215Fab-SEA or C215Fab-SEA and anti-PD-1 mAb therapy following rechallenge with MC38 colon cancer cells. TTS=C215Fab-SEA; and aPD1=anti-PD-1 mAb. FIG. 10A shows initial results after 203 days of the study, and FIG. 10B shows the complete results of the study.

FIGS. 11A-11H are contour plots illustrating proliferation of T cells isolated from the indicated mice in response to incubation with MC38 cancer cells (“MC38”) or MC38-EpCAM cancer cells (“MC38-C215”). FIG. 11A depicts the percentage of proliferating TRBV3 negative CD4 T cells after incubation of T cells isolated from control, naïve mice with MC38-EpCAM cells. FIG. 11B depicts the percentage of proliferating TRBV3 negative CD4 T cells after incubation of T cells isolated from control, tumor-bearing mice with MC38-EpCAM cells. FIG. 11C depicts the percentage of proliferating TRBV3 negative CD4 T cells after incubation of T cells isolated from rechallenged, cured mice (previously treated with C215Fab-SEA and anti-PD-1 mAb as described in Example 2) with MC38-EpCAM cells. FIG. 11D depicts the percentage of proliferating TRBV3 negative CD4 T cells after incubation of T cells isolated from rechallenged, cured mice (previously treated with C215Fab-SEA and anti-PD-1 mAb as described in Example 2) with MC38 cells. FIG. 11E depicts the percentage of proliferating TRBV3 negative CD8 T cells after incubation of T cells isolated from control, naïve mice with MC38-EpCAM cells. FIG. 11F depicts the percentage of proliferating TRBV3 negative CD8 T cells after incubation of T cells isolated from control, tumor-bearing mice with MC38-EpCAM cells. FIG. 11G depicts the percentage of proliferating TRBV3 negative CD8 T cells after incubation of T cells isolated from rechallenged, cured mice (previously treated with C215Fab-SEA and anti-PD-1 mAb as described in Example 2) with MC38-EpCAM cells. FIG. 11H depicts the percentage of proliferating TRBV3 negative CD8 T cells after incubation of T cells isolated from rechallenged, cured mice (previously treated with C215Fab-SEA and anti-PD-1 mAb as described in Example 2) with MC38 cells.

FIGS. 12A-12H are contour plots illustrating proliferation of CD62L-negative effector T cells isolated from the indicated mice in response to incubation with MC38 cancer cells (“MC38”) or MC38-EpCAM cancer cells (“MC38-C215”). FIG. 12A depicts the percentage of proliferating TRBV3 negative CD4 T cells after incubation of T cells isolated from control, naïve mice with MC38-EpCAM cells. FIG. 12B depicts the percentage of proliferating TRBV3 negative CD4 T cells after incubation of T cells isolated from control, tumor-bearing mice with MC38-EpCAM cells. FIG. 12C depicts the percentage of proliferating TRBV3 negative CD4 T cells after incubation of T cells isolated from rechallenged, cured mice (previously treated with C215Fab-SEA and anti-PD-1 mAb as described in Example 2) with MC38-EpCAM cells. FIG. 12D depicts the percentage of proliferating TRBV3 negative CD4 T cells after incubation of T cells isolated from rechallenged, cured mice (previously treated with C215Fab-SEA and anti-PD-1 mAb as described in Example 2) with MC38 cells. FIG. 12E depicts the percentage of proliferating TRBV3 negative CD8 T cells after incubation of T cells isolated from control, naïve mice with MC38-EpCAM cells. FIG. 12F depicts the percentage of proliferating TRBV3 negative CD8 T cells after incubation of T cells isolated from control, tumor-bearing mice with MC38-EpCAM cells. FIG. 12G depicts the percentage of proliferating TRBV3 negative CD8 T cells after incubation of T cells isolated from rechallenged, cured mice (previously treated with C215Fab-SEA and anti-PD-1 mAb as described in Example 2) with MC38-EpCAM cells. FIG. 12H depicts the percentage of proliferating TRBV3 negative CD8 T cells after incubation of T cells isolated from rechallenged, cured mice (previously treated with C215Fab-SEA and anti-PD-1 mAb as described in Example 2) with MC38 cells.

FIGS. 13A-13H are dot plots illustrating the levels of Granzyme B, TNFα and IFNγ in T cells isolated from the indicated mice in response to restimulation and incubation with MC38 cancer cells (“MC38”) or MC38-EpCAM cancer cells (“MC38-C215”). FIG. 13A depicts the percentage of proliferating Granzyme B positive, TRBV3 negative CD8 cytotoxic T cells after T cells isolated from control, naïve mice were restimulated and incubated with MC38-EpCAM cells. FIG. 13B depicts the percentage of proliferating Granzyme B positive, TRBV3 negative CD8 cytotoxic T cells after T cells isolated from control, tumor-bearing mice were restimulated and incubated with MC38-EpCAM cells. FIG. 13C depicts the percentage of proliferating Granzyme B positive, TRBV3 negative CD8 cytotoxic T cells after T cells isolated from rechallenged, cured mice (previously treated with C215Fab-SEA and anti-PD-1 mAb as described in Example 2) were restimulated and incubated with MC38-EpCAM cells. FIG. 13D depicts the percentage of proliferating Granzyme B positive, TRBV3 negative CD8 cytotoxic T cells after T cells isolated from rechallenged, cured mice (previously treated with C215Fab-SEA and anti-PD-1 mAb as described in Example 2) were restimulated and incubated with MC38 cells. FIG. 13E depicts the percentage of TRBV3 negative CD8 T cells producing TNFα and/or IFNγ after T cells isolated from control, naïve mice were restimulated and incubated with MC38-EpCAM cells. FIG. 13F depicts percentage of TRBV3 negative CD8 T cells producing TNFα and/or IFNγ after T cells isolated from control, tumor-bearing mice were restimulated and incubated with MC38-EpCAM cells. FIG. 13G depicts percentage of TRBV3 negative CD8 T cells producing TNFα and/or IFNγ after T cells isolated from rechallenged, cured mice (previously treated with C215Fab-SEA and anti-PD-1 mAb as described in Example 2) were restimulated and incubated with MC38-EpCAM cells. FIG. 13H depicts percentage of TRBV3 negative CD8 T cells producing TNFα and/or IFNγ after T cells isolated from rechallenged, cured mice (previously treated with C215Fab-SEA and anti-PD-1 mAb as described in Example 2) were restimulated and incubated with MC38 cells.

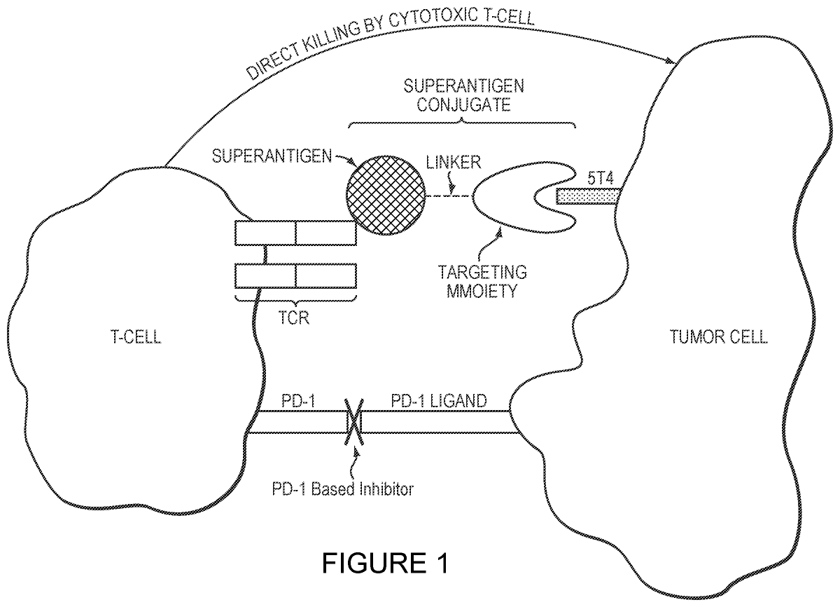

### DETAILED DESCRIPTION

The present invention relates to methods and compositions for treating cancer in a subject. In particular, the invention is based, in part, upon the discovery that a targeted immune response against a cancer in a subject can be significantly enhanced by combining a superantigen conjugate comprising a superantigen covalently linked to a targeting moiety that binds a cancer antigen (e.g., 5T4) with an immunopotentiator, e.g., a PD-1-based inhibitor. It has been discovered that the administration of the superantigen conjugate together with the immunopotentiator can result in enhanced anti-cancer effect for both the superantigen conjugate and the immunopotentiator when combined together (i.e., the agents act synergistically) to produce an effect that is greater than the additive effect of each agent when administered alone.

Furthermore, it has been discovered that administration of a superantigen conjugate comprising a superantigen covalently linked to a targeting moiety that binds a cancer antigen (e.g., 5T4) can promote anticancer immune memory. These results are surprising in particular given that certain other immunotherapies that target T-cells to cancer antigens (e.g., CD3-targeted bispecific antibodies) have not been shown to induce an anticancer immune memory response (see, e.g., Benonisson et al. (2019) MOL. CANCER THER. 18(2): 312-322).

Without wishing to be bound by theory, it is contemplated that the superantigen conjugate may promote epitope spreading, i.e., the superantigen conjugate can elicit an initial immune response in the subject mediated by the superantigen (which is directed to an epitope on the cancer antigen through the superantigen conjugate) which then spreads to one or more immune responses against other epitopes on other cancer antigens. As a result, the superantigen conjugate, optionally in combination with an immunopotentiator, e.g., a PD-1-based inhibitor, can treat a cancer, treat an otherwise refractory cancer, delay recurrence of a cancer, and/or reduce the likelihood of recurrence of a cancer.

In one aspect, the invention provides a method of reducing the likelihood of a recurrence of a cancer in a subject in need thereof. The method comprises administering to the subject: (i) an effective amount of a superantigen conjugate comprising a superantigen covalently linked to a targeting moiety that binds a cancer antigen (e.g., 5T4 or EpCAM) expressed by cancerous cells within the subject; and optionally (ii) an effective amount of an immunopotentiator, e.g., a PD-1 or PD-L1 inhibitor.

In another aspect, the invention provides a method of delaying a recurrence of a cancer in a subject in need thereof. The method comprises administering to the subject: (i) an effective amount of a superantigen conjugate comprising a superantigen covalently linked to a targeting moiety that binds a cancer antigen (e.g., 5T4 or EpCAM) expressed by cancerous cells within the subject; and optionally (ii) an effective amount of an immunopotentiator, e.g., a PD-1 or PD-L1 inhibitor.

In another aspect, the invention provides a method of treating cancer and promoting anticancer immune memory and/or epitope spreading in a subject in need thereof. The method comprises administering to the subject: (i) an effective amount of a superantigen conjugate comprising a superantigen covalently linked to a targeting moiety that binds a cancer antigen (e.g., 5T4 or EpCAM) expressed by cancerous cells within the subject; and optionally (ii) an effective amount of an immunopotentiator, e.g., a PD-1 or PD-L1 inhibitor.

In another aspect, the invention provides a method of promoting anticancer immune memory and/or epitope spreading in a subject with cancer. The method comprises administering to the subject: (i) an effective amount of a superantigen conjugate comprising a superantigen covalently linked to a targeting moiety that binds a cancer antigen (e.g., 5T4 or EpCAM) expressed by cancerous cells within the subject; and optionally (ii) an effective amount of an immunopotentiator, e.g., a PD-1 or PD-L1 inhibitor.

In another aspect, the invention provides a method of inducing at least a first and second epitope-specific immune response in a subject with cancer. The method comprises administering to the subject: (i) an effective amount of a superantigen conjugate comprising a superantigen covalently linked to a targeting moiety that binds a cancer antigen (e.g., 5T4 or EpCAM) expressed by cancerous cells within the subject; and optionally (ii) an effective amount of an immunopotentiator, e.g., a PD-1 or PD-L1 inhibitor, wherein the first epitope-specific immune response is directed to the cancer antigen through the superantigen conjugate and the second epitope-specific immune response is not directed to the cancer antigen or the superantigen and is mediated by epitope spreading.

In another aspect, the invention provides a method of mediating a long term (for example, at least 6 months, 7 months, 8 month, 9 months, 10 months, 11 months, 1 year, 2 years or more) immune response against multiple, different cancer antigens expressed by a cancerous cell in a subject in need of treatment. The method comprises (or consists essentially of) administering to the subject: (i) an effective amount of a superantigen conjugate comprising a superantigen covalently linked to a targeting moiety that binds a single type of cancer antigen expressed by cancerous cells within the subject; and optionally (ii) an effective amount of an immunopotentiator.

In another aspect, the invention provides a method of stimulating an immune response in a subject against a cancerous cell which does not express a 5T4 cancer antigen. The method comprises administering to the subject: (i) an effective amount of a superantigen conjugate comprising a superantigen covalently linked to a targeting moiety that binds a 5T4 cancer antigen expressed by cancerous cells within the subject; and optionally (ii) an effective amount of an immunopotentiator, e.g., a PD-1 or PD-L1 inhibitor. In another aspect, the invention provides a method of stimulating an immune response in a subject against a cancerous cell which does not express an EpCAM cancer antigen. The method comprises administering to the subject: (i) an effective amount of a superantigen conjugate comprising a superantigen covalently linked to a targeting moiety that binds an EpCAM cancer antigen expressed by cancerous cells within the subject; and optionally (ii) an effective amount of an immunopotentiator, e.g., a PD-1 or PD-L1 inhibitor.

In another aspect, the invention provides a method of treating colon or colorectal cancer in a subject in need thereof. The method comprises administering to the subject: (i) an effective amount of a superantigen conjugate comprising a superantigen covalently linked to a targeting moiety that binds a cancer antigen (e.g., 5T4 or EpCAM) expressed by cancerous cells within the subject; and optionally (ii) an effective amount of an immunopotentiator, e.g., a PD-1 or PD-L1 inhibitor.

### I. Definitions

Unless defined otherwise, technical and scientific terms used herein have the same meaning as commonly understood by one of ordinary skill in the art to which this invention belongs. For purposes of the present invention, the following terms are defined below.

As used herein, the terms “a” or “an” may mean one or more. For example, a statement such as “treatment with a superantigen and an immunopotentiator,” can mean treatment: with one superantigen and immunopotentiator; with more than one superantigen and one immunopotentiator; with one superantigen and more than one immunopotentiator; or with more than one superantigen and more than one immunopotentiator.

As used herein, unless otherwise indicated, the term “antibody” is understood to mean an intact antibody (e.g., an intact monoclonal antibody) or antigen-binding fragment of an antibody, including an intact antibody or antigen-binding fragment of an antibody (e.g., a phage display antibody including a fully human antibody, a semisynthetic antibody or a fully synthetic antibody) that has been optimized, engineered or chemically conjugated. Examples of antibodies that have been optimized are affinity-matured antibodies. Examples of antibodies that have been engineered are Fc optimized antibodies, antibodies engineered to reduce immunogenicity, and multi-specific antibodies (e.g., bispecific antibodies). Examples of antigen-binding fragments include Fab, Fab′, F(ab′)2, Fv, single chain antibodies (e.g., scFv), minibodies and diabodies. An antibody conjugated to a toxin moiety is an example of a chemically conjugated antibody.

As used herein, the terms “cancer” and “cancerous” are understood to mean the physiological condition in mammals that is typically characterized by unregulated cell growth. Examples of cancer include, but are not limited to, melanoma, carcinoma, lymphoma, blastoma, sarcoma, and leukemia or lymphoid malignancies. More particular examples of cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, bone cancer, brain cancer, retinoblastoma, endometrial cancer or uterine carcinoma, salivary gland carcinoma, kidney or renal cancer, prostate cancer, vulval cancer, thyroid cancer, hepatic carcinoma, anal carcinoma, penile carcinoma, testicular cancer, as well as head and neck cancer, gum or tongue cancer. The cancer comprises cancer or cancerous cells, for example, the cancer may comprise a plurality of individual cancer or cancerous cells, for example, a leukemia, or a tumor comprising a plurality of associated cancer or cancerous cells.

As used herein, the term “refractory” refers to a cancer that does not respond or no longer responds to a treatment. In certain embodiments, a refractory cancer can be resistant to a treatment before or at the beginning of the treatment. In other embodiments, the refractory cancer can become resistant during or after a treatment. A refractory cancer is also called a resistant cancer. As used herein, the term “recurrence” or “relapse” refers to the return of a refractory cancer or the signs and symptoms of a refractory cancer after a positive response a prior treatment (e.g., a reduction in tumor burden, a reduction in tumor volume, a reduction in tumor metastasis, or a modulation of a biomarker indicative of a positive response to a treatment).

As used herein, the term “immunogen” is a molecule that provokes (evokes, induces, or causes) an immune response. This immune response may involve antibody production, the activation of certain cells, such as, for example, specific immunologically-competent cells, or both. An immunogen may be derived from many types of substances, such as, but not limited to, molecules from organisms, such as, for example, proteins, subunits of proteins, killed or inactivated whole cells or lysates, synthetic molecules, and a wide variety of other agents both biological and nonbiological. It is understood that essentially any macromolecule (including naturally occurring macromolecules or macromolecules produced via recombinant DNA approaches), including virtually all proteins, can serve as immunogens.

As used herein, the term “immunogenicity” relates to the ability of an immunogen to provoke (evoke, induce, or cause) an immune response. Different molecules may have differing degrees of immunogenicity, and a molecule having an immunogenicity that is greater compared to another molecule is known, for example, to be capable of provoking (evoking, inducing, or causing) a greater immune response than would an agent having a lower immunogenicity.

As used herein, the term “antigen” as used herein refers to a molecule that is recognized by antibodies, specific immunologically-competent cells, or both. An antigen may be derived from many types of substances, such as, but not limited to, molecules from organisms, such as, for example, proteins, subunits of proteins, nucleic acids, lipids, killed or inactivated whole cells or lysates, synthetic molecules, and a wide variety of other agents both biological and non-biological.

As used herein, the term “antigenicity” relates to the ability of an antigen to be recognized by antibodies, specific immunologically-competent cells, or both.

As used herein, the term “epitope spreading” refers to the diversification of the epitope specificity of an immune response from an initial epitope-specific immune response directed against an antigen to other epitopes on that antigen (intramolecular spreading) or other antigens (intermolecular spreading). Epitope spreading allows a subject's immune system to determine additional target epitopes not initially recognized by the immune system in response to the original therapeutic protocol while reducing the possibility of escape variants in a tumor population and thus affect progression of disease.

As used herein, the term “immune response” refers to a response by a cell of the immune system, such as a B cell, T cell (CD4+ or CD8+), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), and refers to a response by a CD4+ T cell, CD8+ T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response.

As used herein, the term “major histocompatibility complex,” or “MHC,” refers to a specific cluster of genes, many of which encode evolutionarily related cell surface proteins involved in antigen presentation, that are important determinants of histocompatibility. Class I MHC, or MHC-I, function mainly in antigen presentation to CD8+ T lymphocytes (CD8+ T-Cells). Class II MHC, or MHC-II, function mainly in antigen presentation to CD4+ T lymphocytes (CD4+ T-Cells).

As used herein, the term “derived,” for example “derived from,” includes, but is not limited to, for example, wild-type molecules derived from biological hosts such as bacteria, viruses and eukaryotic cells and organisms, and modified molecules, for example, modified by chemical means or produced in recombinant expression systems.

As used herein, the terms “seroreactive,” “seroreaction” or “seroreactivity” are understood to mean the ability of an agent, such as a molecule, to react with antibodies in the serum of a mammal, such as, but not limited to, a human. This includes reactions with all types of antibodies, including, for example, antibodies specific for the molecule and nonspecific antibodies that bind to the molecule, regardless of whether the antibodies inactivate or neutralize the agent. As is known in the art, different agents may have different seroreactivity relative to one another, wherein an agent having a seroreactivity lower than another would, for example, react with fewer antibodies and/or have a lower affinity and/or avidity to antibodies than would an agent having a higher seroreactivity. This may also include the ability of the agent to elicit an antibody immune response in an animal, such as a mammal, such as a human.

As used herein, the terms “soluble T-cell receptor,” or “soluble TCR,” are understood to mean a “soluble” T-cell receptor comprising the chains of a full-length (e.g., membrane bound) receptor, except that the transmembrane region of the receptor chains are deleted or mutated so that the receptor, when expressed by a cell, will not insert into, traverse or otherwise associate with the membrane. A soluble T-cell receptor may comprise only the extracellular domains or extracellular fragments of the domains of the wild-type receptor (e.g., lacks the transmembrane and cytoplasmic domains).

As used herein, the term “superantigen” is understood to mean a class of molecules that stimulate a subset of T-cells by binding to MHC class II molecules and Vβ domains of T-cell receptors, thereby activating T-cells expressing particular Vβ gene segments. The term includes wild-type, naturally occurring superantigens, for example, those isolated from certain bacteria or expressed from unmodified genes from same, as well as modified superantigens, wherein, for example, the DNA sequence encoding a superantigen has been modified, for example, by genetic engineering, to, for example, produce a fusion protein with a targeting moiety, and/or alter certain properties of the superantigen, such as, but not limited to, its MHC class II binding (for example, to reduce affinity) and/or its seroreactivity, and/or its immunogenicity, and/or antigenicity (for example, to reduce its seroreactivity). The definition includes wild-type and modified superantigens and any immunologically reactive variants and/or fragments thereof described herein or in the following U.S. patents and patent applications: U.S. Pat. Nos. 5,858,363, 6,197,299, 6,514,498, 6,713,284, 6,692,746, 6,632,640, 6,632,441, 6,447,777, 6,399,332, 6,340,461, 6,338,845, 6,251,385, 6,221,351, 6,180,097, 6,126,945, 6,042,837, 6,713,284, 6,632,640, 6,632,441, 5,859,207, 5,728,388, 5,545,716, 5,519,114, 6,926,694, 7,125,554, 7,226,595, 7,226,601, 7,094,603, 7,087,235, 6,835,818, 7,198,398, 6,774,218, 6,913,755, 6,969,616, and 6,713,284, U.S. Patent Application Nos. 2003/0157113, 2003/0124142, 2002/0177551, 2002/0141981, 2002/0115190, 2002/0051765, and 2001/0046501, and PCT International Publication Number WO/03/094846.

As used herein, the term “targeting moiety” refers to any structure, molecule or moiety that is able to bind to a cellular molecule, for example, a cell surface molecule, preferably a disease specific molecule such as an antigen expressed preferentially on a cancer (or cancerous) cell. Exemplary targeting moieties include, but are not limited to, antibodies (including antigen binding fragments thereof) and the like, soluble T-cell receptors, interleukins, hormones, and growth factors.

As used herein, the terms “tumor-targeted superantigen” or “TTS” or “cancer-targeted superantigen” are understood to mean a molecule comprising one or more superantigens covalently linked (either directly or indirectly) with one or more targeting moieties.

As used herein, the term “T-cell receptor” is understood to mean a receptor that is specific to T-cells, and includes the understanding of the term as known in the art. The term also includes, for example, a receptor that comprises a disulfide-linked heterodimer of the highly variable α or β chains expressed at the cell membrane as a complex with the invariant CD3 chains, and a receptor made up of variable γ and δ chains expressed at the cell membrane as a complex with CD3 on a subset of T-cells.

As used herein, the terms “therapeutically effective amount” and “effective amount,” are understood to mean an amount of an active agent, for example, a pharmaceutically active agent or a pharmaceutical composition that produces at least some effect in treating a disease or a condition. The effective amount of pharmaceutically active agent(s) used to practice the present invention for a therapeutic treatment varies depending upon the manner of administration, the age, body weight, and general health of the subject. These terms include, but are not limited to synergistic situations such as those described in the instant invention wherein a single agent alone, such as a superantigen conjugate or a PD-1-based inhibitor (for example, an anti-PD-1 antibody), may act weakly or not at all, but when combined with each other, for example, but not limited to, via sequential dosage, the two or more agents act to produce a synergistic result.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

As used herein, the terms “treat,” “treating” and “treatment” are understood to mean the treatment of a disease in a mammal, e.g., in a human. This includes: (a) inhibiting the disease, i.e., arresting its development; and (b) relieving the disease, i.e., causing regression of the disease state; and (c) curing the disease. As used in the context of a therapeutic treatment, the terms “prevent” or “block” are understood to completely prevent or block, or not completely prevent or block (e.g., partially prevent or block) a given act, action, activity, or event.

As used herein, the term “inhibits the growth of a cancer” is understood to mean a measurably slowing, stopping, or reversing the growth rate of the cancer or cancerous cells in vitro or in vivo. Desirably, the growth rate is slowed by 20%, 30%, 50%, or 70% or more, as determined using a suitable assay for determination of cell growth rates. Typically, a reversal of growth rate is accomplished by initiating or accelerating necrotic or apoptotic mechanisms of cell death in neoplastic cells, resulting in a shrinkage of a neoplasm.

As used herein, the terms “variant,” “variants,” “modified,” “altered,” “mutated,” and the like, are understood to mean proteins or peptides and/or other agents and/or compounds that differ from a reference protein, peptide or other compound. Variants in this sense are described below and elsewhere in greater detail. For example, changes in a nucleic acid sequence of the variant may be silent, e.g., they may not alter the amino acids encoded by the nucleic acid sequence. Where alterations are limited to silent changes of this type a variant will encode a peptide with the same amino acid sequence as the reference peptide. Changes in the nucleic acid sequence of the variant may alter the amino acid sequence of a peptide encoded by the reference nucleic acid sequence. Such nucleic acid changes may result in amino acid substitutions, additions, deletions, fusions and/or truncations in the protein or peptide encoded by the reference sequence, as discussed below. Generally, differences in amino acid sequences are limited so that the sequences of the reference and the variant are similar overall and, in many regions, identical. A variant and reference protein or peptide may differ in amino acid sequence by one or more substitutions, additions, deletions, fusions and/or truncations, which may be present in any combination. A variant may also be a fragment of a protein or peptide of the invention that differs from a reference protein or peptide sequence by being shorter than the reference sequence, such as by a terminal or internal deletion. Another variant of a protein or peptide of the invention also includes a protein or peptide which retains essentially the same function or activity as the reference protein or peptide. A variant may also be: (i) one in which one or more of the amino acid residues are substituted with a conserved or non-conserved amino acid residue and such substituted amino acid residue may or may not be one encoded by the genetic code, or (ii) one in which one or more of the amino acid residues includes a substituent group, or (iii) one in which the mature protein or peptide is fused with another compound, such as a compound to increase the half-life of the protein or peptide (for example, polyethylene glycol), or (iv) one in which the additional amino acids are fused to the mature protein or peptide, such as a leader or secretory sequence or a sequence which is employed for purification of the mature protein or peptide. Variants may be made by mutagenesis techniques, and/or altering mechanisms such as chemical alterations, fusions, adjuncts and the like, including those applied to nucleic acids, amino acids, cells or organisms, and/or may be made by recombinant means.

As used herein, the term “sequential dosage” and related terminology refers to the administration of at least one superantigen, with at least one PD-1-based inhibitor, and includes staggered doses of these agents (i.e., time-staggered) and variations in dosage amounts. This includes one agent being administered before, overlapping with (partially or totally), or after administration of another agent. This term generally considers the best administration scheme to achieve a synergistic combination of at least one superantigen and at least one PD-1-based inhibitor. By such a dosing strategy (e.g., a sequential dosage), one may be able to achieve synergistic effects of combined superantigen and PD-1-based inhibitor administration. In addition, the term “sequential dosage” and related terminology also includes the administration of at least one superantigen, one PD-1-based inhibitor and more or more optional additional compounds such as, for example, a corticosteroid, an immune modulator, and another agent designed to reduce potential immunoreactivity to the superantigen conjugate administered to the subject.

As used herein, the terms “systemic” and “systemically” in the context of administration are understood to mean administration of an agent such that the agent is exposed to at least one system associated with the whole body, such as but not limited to the circulatory system, immune system, and lymphatic system, rather than only to a localized part of the body, such as but not limited to within a tumor. Thus, for example, a systemic therapy or an agent administered systematically is a therapy or an agent in which at least one system associated with the entire body is exposed to the therapy or agent, as opposed to, rather than just a target tissue.

As used herein, the term “parenteral administration” includes any form of administration in which the compound is absorbed into the subject without involving absorption via the intestines. Exemplary parenteral administrations that are used in the present invention include, but are not limited to intramuscular, intravenous, intraperitoneal, or intraarticular administration.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

At various places in the present specification, values are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing and method steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### II. Superantigen Conjugate

A. Superantigens

Superantigens are bacterial proteins, viral proteins, and human-engineered proteins, capable of activating T lymphocytes, for example, at picomolar concentrations. Superantigens can also activate large subsets of T lymphocytes (T-cells). Superantigens can bind to the major histocompatibility complex I (MHCI) without being processed and, in particular, can bind to conserved regions outside the antigen-binding groove on MHC class II molecules, avoiding most of the polymorphism in the conventional peptide-binding site. Superantigens can also bind to the Vβ chain of the T-cell receptor (TCR) rather than binding to the hypervariable loops of the T-cell receptor. Examples of bacterial superantigens include, but are not limited to, Staphylococcal enterotoxin (SE), Streptococcus pyogenes exotoxin (SPE), Staphylococcus aureus toxic shock-syndrome toxin (TSST-1), Streptococcal mitogenic exotoxin (SME), Streptococcal superantigen (SSA), Staphylococcal enterotoxin A (SEA), Staphylococcal enterotoxin A (SEB), and Staphylococcal enterotoxin E (SEE).

The polynucleotide sequences encoding many superantigens have been isolated and cloned and superantigens expressed from these or modified (reengineered) polynucleotide sequences have been used in anti-cancer therapy (see, naptumomab estafenatox/ANYARA®, discussed below). Superantigens expressed by these polynucleotide sequences may be wild-type superantigens, modified superantigens, or wild-type or modified superantigens conjugated or fused with targeting moieties. The superantigens may be administered to a mammal, such as a human, directly, for example by injection, or may be delivered, for example, by exposure of blood of a patient to the superantigen outside the body, or, for example, via placing a gene encoding a superantigen inside a mammal to be treated (e.g., via known gene therapy methods and vectors such as, for example, via cells containing, and capable of expressing, the gene) and expressing the gene within the mammal.

Examples of superantigens and their administration to mammals are described in the following U.S. patents and patent applications: U.S. Pat. Nos. 5,858,363, 6,197,299, 6,514,498, 6,713,284, 6,692,746, 6,632,640, 6,632,441, 6,447,777, 6,399,332, 6,340,461, 6,338,845, 6,251,385, 6,221,351, 6,180,097, 6,126,945, 6,042,837, 6,713,284, 6,632,640, 6,632,441, 5,859,207, 5,728,388, 5,545,716, 5,519,114, 6,926,694, 7,125,554, 7,226,595, 7,226,601, 7,094,603, 7,087,235, 6,835,818, 7,198,398, 6,774,218, 6,913,755, 6,969,616, and 6,713,284, U.S. Patent Application Nos. 2003/0157113, 2003/0124142, 2002/0177551, 2002/0141981, 2002/0115190, and 2002/0051765, and PCT International Publication Number WO/03/094846.

B. Modified Superantigens

Within the scope of this invention, superantigens may be engineered in a variety of ways, including modifications that retain or enhance the ability of a superantigen to stimulate T lymphocytes, and may, for example, alter other aspects of the superantigen, such as, for example, its seroreactivity or immunogenicity. Modified superantigens include synthetic molecules that have superantigen activity (i.e., the ability to activate subsets of T lymphocytes).

It is contemplated that various changes may be made to the polynucleotide sequences encoding a superantigen without appreciable loss of its biological utility or activity, namely the induction of the T-cell response to result in cytotoxicity of the tumor cells. Furthermore, the affinity of the superantigen for the MHC class II molecule can be decreased with minimal effects on the cytotoxicity of the superantigen. This, for example, can help to reduce toxicity that may otherwise occur if a superantigen retains its wild-type ability to bind MHC class II antigens (as in such a case, class II expressing cells, such as immune system cells, could also be affected by the response to the superantigen).

Techniques for modifying superantigens (e.g., polynucleotides and polypeptides), including for making synthetic superantigens, are well known in the art and include, for example PCR mutagenesis, alanine scanning mutagenesis, and site-specific mutagenesis (see, U.S. Pat. Nos. 5,220,007; 5,284,760; 5,354,670; 5,366,878; 5,389,514; 5,635,377; and 5,789,166).

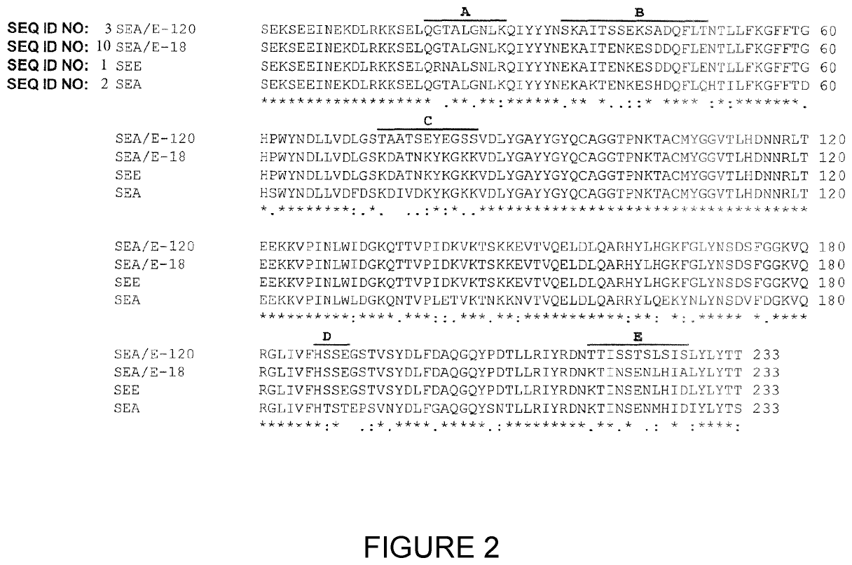

In some embodiments, a superantigen may be modified such that its seroreactivity is reduced compared to a reference wild-type superantigen, but its ability to activate T-cells is retained or enhanced relative to wild-type. One technique for making such modified superantigens includes substituting certain amino acids in certain regions from one superantigen to another. This is possible because many superantigens, including but not limited to, SEA, SEE, and SED, share sequence homology in certain areas that have been linked to certain functions (Marrack and Kappler (1990) SCIENCE 248(4959): 1066; see also FIG. 2, which shows region of homology between different wild type and engineered superantigens). For example, in certain embodiments of the present invention, a superantigen that has a desired T-cell activation-inducing response, but a non-desired high seroreactivity, is modified such that the resulting superantigen retains its T-cell activation ability but has reduced seroreactivity.

It is known and understood by those of skill in the art that the sera of humans normally contain various titers of antibodies against superantigens. For the staphylococcal superantigens, for instance, the relative titers are TSST-1>SEB>SEC-1>SE3>SEC2>SEA>SED>SEE. As a result, the seroreactivity of, for example, SEE (Staphylococcal enterotoxin E) is lower than that of, for example, SEA (Staphylococcal enterotoxin A). Based on this data, one skilled in the art may prefer to administer a low titer superantigen, such as, for example SEE, instead of a high titer superantigen, such as, for example, SEB (Staphylococcal enterotoxin B). However, as has also been discovered, different superantigens have differing T-cell activation properties relative to one another, and for wild-type superantigens, the best T-cell activating superantigens often also have undesirably high seroreactivity.

These relative titers sometimes correspond to potential problems with seroreactivity, such as problems with neutralizing antibodies. Thus, the use of a low titer superantigen, such as SEA or SEE may be helpful in reducing or avoiding seroreactivity of parenterally administered superantigens. A low titer superantigen has a low seroreactivity as measured, for example, by typical anti-superantigen antibodies in a general population. In some instances it may also have a low immunogenicity. Such low titer superantigens may be modified to retain its low titer as described herein.

Approaches for modifying superantigens can be used to create superantigens that have both the desired T-cell activation properties and reduced seroreactivity, and in some instances also reduced immunogenicity. Given that certain regions of homology between superantigens relate to seroreactivity, it is possible to engineer a recombinant superantigen that has a desired T-cell activation and a desired seroreactivity and/or immunogenicity. Furthermore, the protein sequences and immunological cross-reactivity of the superantigens or staphylococcal enterotoxins are divided into two related groups. One group consists of SEA, SEE and SED. The second group is SPEA, SEC and SEB. Thus, it is possible to select low titer superantigens to decrease or eliminate the cross-reactivity with high titer or endogenous antibodies directed against staphylococcal enterotoxins.

Regions in the superantigens that are believed to play a role in seroreactivity include, for example, Region A, which comprises amino acid residues 20, 21, 22, 23, 24, 25, 26, and 27; Region B, which comprises amino acid residues 34, 35, 36, 37, 38, 39, 40, 41, 42, 43, 44, 45, 46, 47, 48, and 49; Region C, which comprises amino acid residues 74, 75, 76, 77, 78, 79, 80, 81, 82, 83, and 84; Region D, which comprises amino acid residues 187, 188, 189 and 190; and Region E, which comprise the amino acid residues, 217, 218, 219, 220, 221, 222, 223, 224, 225, 226, and 227 (see, U.S. Pat. No. 7,125,554, and FIG. 2 herein). Thus, it is contemplated that these regions can be mutated using, for example amino acid substitution, to produce a superantigen having altered seroreactivity.

Polypeptide or amino acid sequences for the above listed superantigens can be obtained from any sequence data bank, for example Protein Data Bank and/or GenBank. Exemplary GenBank accession numbers include, but are not limited to, SEE is P12993; SEA is P013163; SEB is P01552; SECT is P01553; SED is P20723; and SEH is AAA19777.

In certain embodiments of the present invention, the wild-type SEE sequence (SEQ ID NO: 1) or the wild type SEA sequence (SEQ ID NO: 2) can be modified such that amino acids in any of the identified regions A-E (see, FIG. 2) are substituted with other amino acids. Such substitutions include for example, K79, K81, K83 and D227 or K79, K81, K83, K84 and D227, or, for example, K79E, K81E, K83S and D227S or K79E, K81E, K83S, K84S and D227A. In certain embodiments, the superantigen is SEA/E-120 (SEQ ID NO: 3; see also U.S. Pat. No. 7,125,554) or SEA_{D227A} (SEQ ID NO: 4; see also U.S. Pat. No. 7,226,601).

1. Modified Polynucleotides and Polypeptides

A biological functional equivalent of a polynucleotide encoding a naturally occurring or a reference superantigen may comprise a polynucleotide that has been engineered to contain distinct sequences while at the same time retaining the capacity to encode the naturally occurring or reference superantigen. This can be accomplished due to the degeneracy of the genetic code, i.e., the presence of multiple codons, which encode for the same amino acids. In one example, it is possible to introduce a restriction enzyme recognition sequence into a polynucleotide while not disturbing the ability of that polynucleotide to encode a protein. Other polynucleotide sequences may encode superantigens that are different but functionally substantially equivalent in at least one biological property or activity (for example, at least 50%, 60%, 70%, 80%, 90%, 95%, 98% of the biological property or activity, for example, without limitation, the ability to induce a T-cell response that results in cytotoxicity of the tumor cells) to a reference superantigen.

In another example, a polynucleotide may be (and encode) a superantigen functionally equivalent to a reference superantigen even though it may contain more significant changes. Certain amino acids may be substituted for other amino acids in a protein structure without appreciable loss of interactive binding capacity with structures such as, for example, antigen-binding regions of antibodies, binding sites on substrate molecules, receptors, and such like. Furthermore, conservative amino acid replacements may not disrupt the biological activity of the protein, as the resultant structural change often is not one that impacts the ability of the protein to carry out its designed function. It is thus contemplated that various changes may be made in the sequence of genes and proteins disclosed herein, while still fulfilling the goals of the present invention.

Amino acid substitutions may be designed to take advantage of the relative similarity of the amino acid side-chain substituents, for example, their hydrophobicity, hydrophilicity, charge, size, and/or the like. An analysis of the size, shape and/or type of the amino acid side-chain substituents reveals that arginine, lysine and/or histidine are all positively charged residues; that alanine, glycine and/or serine are all a similar size; and/or that phenylalanine, tryptophan and/or tyrosine all have a generally similar shape. Therefore, based upon these considerations, arginine, lysine and/or histidine; alanine, glycine and/or serine; and/or phenylalanine, tryptophan and/or tyrosine; are defined herein as biologically functional equivalents. In addition, it may be possible to introduce non-naturally occurring amino acids. Approaches for making amino acid substitutions with other naturally occurring and non-naturally occurring amino acid are described in U.S. Pat. No. 7,763,253.

In terms of functional equivalents, it is understood that, implicit in the definition of a “biologically functional equivalent” protein and/or polynucleotide, is the concept that there is a limited number of changes that may be made within a defined portion of the molecule while retaining a molecule with an acceptable level of equivalent biological activity. Biologically functional equivalents are thus considered to be those proteins (and polynucleotides) where selected amino acids (or codons) may be substituted without substantially affecting biological function. Functional activity includes the induction of the T-cell response to result in cytotoxicity of the tumor cells.

In addition, it is contemplated that a modified superantigen can be created by substituting homologous regions of various proteins via “domain swapping,” which involves the generation of chimeric molecules using different but, in this case, related polypeptides. By comparing various superantigen proteins to identify functionally related regions of these molecules (see, e.g., FIG. 2), it is possible to swap related domains of these molecules so as to determine the criticality of these regions to superantigen function. These molecules may have additional value in that these “chimeras” can be distinguished from natural molecules, while possibly providing the same function.

In certain embodiments, the superantigen comprises an amino acid sequence that is at least 70%, 75%, 80%, 85%, 90%, 95%, 98%, or 99% identical to the sequence of a reference superantigen selected from SEQ ID NO: 1, SEQ ID NO: 2, SEQ ID NO: 3, and SEQ ID NO: 4, wherein the superantigen optionally retains at least 50%, 60%, 70% 80%, 90%, 95%. 98%, 99%, or 100% of a biological activity or property of the reference superantigen.

In certain embodiments, the superantigen comprises an amino acid sequence that is encoded by a nucleic acid that is at least 70%, 75%, 80%, 85%, 90%, 95%, 98%, or 99% identical to a nucleic acid encoding the superantigen selected from SEQ ID NO: 1, SEQ ID NO: 2, SEQ ID NO: 3, and SEQ ID NO: 4, wherein the superantigen optionally retains at least 50%, 60%, 70% 80%, 90%, 95%. 98%, 99%, or 100% of a biological activity or property of the reference superantigen.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36, 290-300; Altschul et al., (1997) NUCLEIC ACIDS RES. 25:3389-3402, incorporated by reference) are tailored for sequence similarity searching. For a discussion of basic issues in searching sequence databases see Altschul et al., (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: −G, Cost to open gap [Integer]: default=5 for nucleotides/11 for proteins; −E, Cost to extend gap [Integer]: default=2 for nucleotides/1 for proteins; −q, Penalty for nucleotide mismatch [Integer]: default=−3; −r, reward for nucleotide match [Integer]: default=1; −e, expect value [Real]: default=10; —W, wordsize [Integer]: default=11 for nucleotides/28 for megablast/3 for proteins; −y, Dropoff (X) for blast extensions in bits: default=20 for blastn/7 for others; −X, X dropoff value for gapped alignment (in bits): default=15 for all programs, not applicable to blastn; and —Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

C. Targeted Superantigens

In order to increase specificity, the superantigen preferably is conjugated to a targeting moiety to create a targeted superantigen conjugate that binds an antigen preferentially expressed by a cancer cell, for example, a cell surface antigen such as 5T4. The targeting moiety is a vehicle that can be used to bind superantigen to the cancerous cells, for example, the surface of the cancerous cells. The targeted superantigen conjugate should retain the ability to activate large numbers of T lymphocytes. For example, the targeted superantigen conjugate should activate large numbers of T-cells and direct them to tissues containing the tumor-associated antigen bound to the targeting moiety. In such situations, specific target cells are preferentially killed, leaving the rest of the body relatively unharmed. This type of therapy is desirable, as non-specific anti-cancer agents, such as cytostatic chemotherapeutic drugs, are nonspecific and kill large numbers of cells not associated with tumors to be treated. For example, studies with targeted superantigen conjugates have shown that inflammation with infiltration by cytotoxic T lymphocytes (CTLs) into tumor tissue increases rapidly in response to the first injection of a targeted superantigen (Dohlsten et al. (1995) PROC. NATL. ACAD. SCI. USA 92:9791-9795). This inflammation with infiltration of CTLs into the tumor is one of the major effectors of the anti-tumor therapeutic of targeted superantigens.

Tumor-targeted superantigens represent an immunotherapy against cancer and are therapeutic fusion proteins containing a targeting moiety conjugated to a superantigen (Dohlsten et al. (1991) PROC. NATL. ACAD. SCI. USA 88:9287-9291; Dohlsten et al. (1994) PROC. NATL. ACAD. SCI. USA 91:8945-8949).

The targeting moiety can in principle be any structure that is able to bind to a cellular molecule, for example, a cell surface molecule and preferably is a disease specific molecule. The targeted molecule (e.g., antigen) against which the targeting moiety is directed is usually different from (a) the Vβ chain epitope to which superantigen binds, and (b) the MHC class II epitopes to which superantigens bind. The targeting moiety can be selected from antibodies, including antigen binding fragments thereof, soluble T-cell receptors, growth factors, interleukins (e.g., interleukin-2), hormones, etc.

In certain preferred embodiments, the targeting moiety is an antibody (e.g., Fab, F(ab)₂, Fv, single chain antibody, etc.). Antibodies are extremely versatile and useful cell-specific targeting moieties because they typically can be generated against any cell surface antigen of interest. Monoclonal antibodies have been generated against cell surface receptors, tumor-associated antigens, and leukocyte lineage-specific markers such as CD antigens. Antibody variable region genes can be readily isolated from hybridoma cells by methods well known in the art. Exemplary tumor-associated antigens that can be used to produce a targeting moiety can include, but are not limited to gp100, Melan-A/MART, MAGE-A, MAGE (melanoma antigen E), MAGE-3, MAGE-4, MAGEA3, tyrosinase, TRP2, NY-ESO-1, CEA (carcinoembryonic antigen), PSA, p53, Mammaglobin-A, Survivin, MUC1 (mucin1)/DF3, metallopanstimulin-1 (MPS-1), Cytochrome P450 isoform 1B1, 90K/Mac-2 binding protein, Ep-CAM (MK-1), HSP-70, hTERT (TRT), LEA, LAGE-1/CAMEL, TAGE-1, GAGE, 5T4, gp70, SCP-1, c-myc, cyclin B1, MDM2, p62, Koc, IMP1, RCAS1, TA90, OA1, CT-7, HOM-MEL-40/SSX-2, SSX-1, SSX-4, HOM-TES-14/SCP-1, HOM-TES-85, HDAC5, MBD2, TRIP4, NY-CO-45, KNSL6, HIP1R, Seb4D, KIAA1416, IMP1, 90K/Mac-2 binding protein, MDM2, NY/ESO, EGFRvIII, IL-13Rα2, HER2, GD2, EGFR, PDL1, Mesothelin, PSMA, TGFβRDN, LMP1, GPC3, Fra, MG7, CD133, CMET, PSCA, Glypican3, ROR1, NKR-2, CD70 and LMNA.

Exemplary cancer-targeting antibodies can include, but are not limited to, anti-CD19 antibodies, anti-CD20 antibodies, anti-5T4 antibodies, anti-Ep-CAM antibodies, anti-Her-2/neu antibodies, anti-EGFR antibodies, anti-CEA antibodies, anti-prostate specific membrane antigen (PSMA) antibodies, and anti-IGF-1R antibodies. It is understood that the superantigen can be conjugated to an immunologically reactive antibody fragment such as C215Fab, 5T4Fab (see, WO8907947) or C242Fab (see, WO9301303).

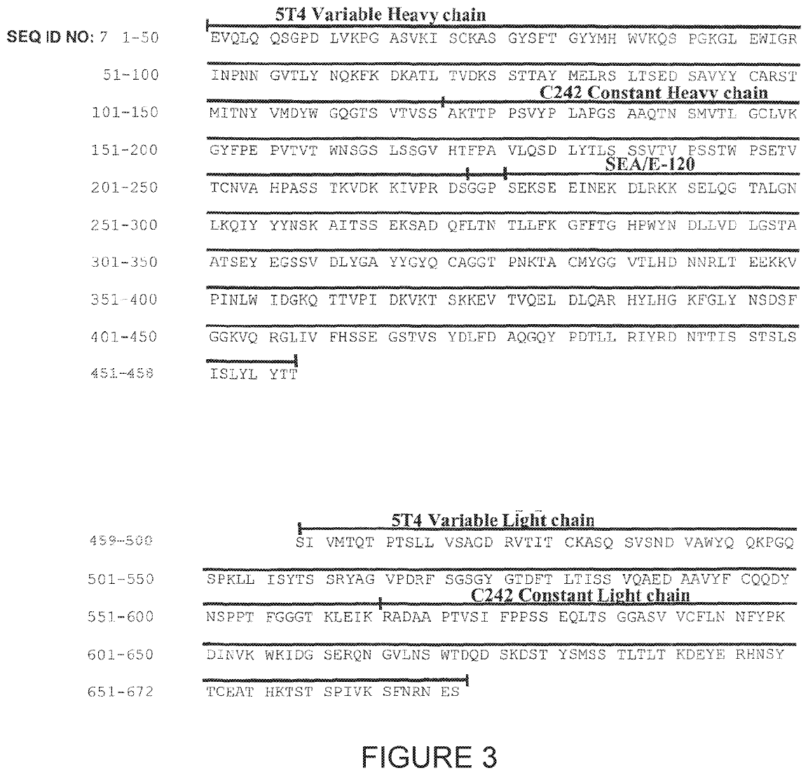

Examples of tumor targeted superantigens that can be used in the present invention include C215Fab-SEA (SEQ ID NO: 5), 5T4Fab-SEA_{D227A} (SEQ ID NO: 6) and 5T4Fab-SEA/E-120 (SEQ ID NO: 7, see FIG. 2 and FIG. 3).

In a preferred embodiment, a preferred conjugate is a superantigen conjugate known as naptumomab estafenatox/ANYARA®, which is the fusion protein of the Fab fragment of an anti-5T4 antibody and the SEA/E-120 superantigen. Naptumomab estafenatox/ANYARA® comprises two protein chains that cumulatively include an engineered Staphylococcal enterotoxin superantigen (SEA/E-120) and a targeting 5T4 Fab comprising modified 5T4 variable region sequences fused to the constant region sequences of the murine IgG1/K antibody C242. The first protein chain comprises residues 1 to 458 of SEQ ID NO: 7 (see also, SEQ ID NO: 8), and includes a chimeric 5T4 Fab heavy chain, corresponding to residues 1 to 222 of SEQ ID NO: 7, and the SEA/E-120 superantigen, corresponding to residues 226 to 458 of SEQ ID NO: 7, covalently linked via a GGP tripeptide linker, corresponding to residues 223-225 of SEQ ID NO: 7. The second chain comprises residues 459 to 672 of SEQ ID NO: 7 (see also, SEQ ID NO: 9) and includes a chimeric 5T4 Fab light chain. The two protein chains are held together by non-covalent interactions between the Fab heavy and light chains. Residues 1-458 of SEQ ID NO: 7 correspond to residues 1-458 of SEQ ID NO: 8, and residues 459-672 of SEQ ID NO: 7 correspond to residues 1-214 of SEQ ID NO: 9. Naptumomab estafenatox/ANYARA® comprises the proteins of SEQ ID NOS: 8 and 9 held together by non-covalent interactions between the Fab heavy and Fab light chains. Naptumomab estafenatox/ANYARA® induces T-cell mediated killing of cancer cells at concentrations around 10 pM and the superantigen component of the conjugate has been engineered to have low binding to human antibodies and MHC Class II.

It is contemplated that other antibody based targeting moieties can be designed, modified, expressed, and purified using techniques known in the art and discussed in more detail below.

Another type of targeting moiety includes a soluble T-cell receptor (TCR). Some forms of soluble TCR may contain either only extracellular domains or extracellular and cytoplasmic domains. Other modifications of the TCR may also be envisioned to produce a soluble TCR in which the transmembrane domains have been deleted and/or altered such that the TCR is not membrane bound as described in U.S. Publication Application Nos. U.S. 2002/119149, U.S. 2002/0142389, U.S. 2003/0144474, and U.S. 2003/0175212, and International Publication Nos. WO2003020763; WO9960120 and WO9960119.

The targeting moiety can be conjugated to the superantigen by using either recombinant techniques or chemically linking of the targeting moiety to the superantigen.

1. Recombinant Linker (Fusion Protein)

It is contemplated that a gene encoding a superantigen linked directly or indirectly (for example, via an amino acid containing linker) to a targeting moiety can be created and expressed using conventional recombinant DNA technologies. For example, the amino terminal of a modified superantigen can be linked to the carboxy terminal of a targeting moiety or vice versa. For antibodies, or antibody fragments that may serve as targeting moieties, either the light or the heavy chain may be utilized for creating a fusion protein. For example, for a Fab fragment, the amino terminus of the modified superantigen can be linked to the first constant domain of the heavy antibody chain (CHO. In some instances, the modified superantigen can be linked to a Fab fragment by linking the VH and VL domain to the superantigen. Alternatively, a peptide linker can be used to join the superantigen and targeting moiety together. When a linker is employed, the linker preferably contains hydrophilic amino acid residues, such as Gln, Ser, Gly, Glu, Pro, His and Arg. Preferred linkers are peptide bridges consisting of 1-10 amino acid residues, more particularly, 3-7 amino acid residues. An exemplary linker is the tripeptide -GlyGlyPro-. These approaches have been used successfully in the design and manufacture of the naptumomab estafenatox/ANYARA® superantigen conjugate.

2. Chemical Linkage

It is also contemplated that the superantigen may be linked to the targeting moiety via a chemical linkage. Chemical linkage of the superantigen to the targeting moiety may require a linker, for example, a peptide linker. The peptide linker preferably is hydrophilic and exhibits one or more reactive moieties selected from amides, thioethers, disulfides etc. (See, U.S. Pat. Nos. 5,858,363, 6,197,299, and 6,514,498). It is also contemplated that the chemical linkage can use homo- or heterobifunctional crosslinking reagents. Chemical linking of a superantigen to a targeting moiety often utilizes functional groups (e.g., primary amino groups or carboxy groups) that are present in many positions in the compounds.

D. Expression of Superantigens and Superantigen Conjugates

When recombinant DNA technologies are employed, the superantigen or the superantigen-targeting moiety conjugate may be expressed using standard expression vectors and expression systems. The expression vectors, which have been genetically engineered to contain the nucleic acid sequence encoding the superantigen, are introduced (e.g., transfected) into host cells to produce the superantigen (see, e.g. Dohlsten et al. (1994), Forsberg et al. (1997) J. BIOL. CHEM. 272:12430-12436, Erlandsson et al. (2003) J. MOL. BIOL. 333:893-905 and WO2003002143).

Host cells can be genetically engineered, for example, by transformation or transfection technologies, to incorporate nucleic acid sequences and express the superantigen. Introduction of nucleic acid sequences into the host cell can be affected by calcium phosphate transfection, DEAE-dextran mediated transfection, microinjection, cationic lipid-mediated transfection, electroporation, transduction, scrape loading, ballistic introduction, infection or other methods. Such methods are described in many standard laboratory manuals, such as, Davis et al. (1986) BASIC METHODS IN MOLECULAR BIOLOGY and Sambrook, et al. (1989) MOLECULAR CLONING: A LABORATORY MANUAL, 2nd Ed., Cold Spring Harbor Laboratory Press, Cold Spring Harbor, N.Y.

Representative examples of appropriate host cells include bacterial cells, such as streptococci, staphylococci, E. coli, Streptomyces and Bacillus subtilis cells; fungal cells, such as yeast cells and aspergillus cells; insect cells such as Drosophila S2 and Spodoptera Sf9 cells; mammalian cells such as CHO, COS, HeLa, C127, 3T3, BHK, HEK-293 and Bowes melanoma cells.

Examples of production systems for superantigens are found, for example, in U.S. Pat. No. 6,962,694.

E. Protein Purification

The superantigen and/or the superantigen-targeting moiety conjugates preferably are purified prior to use, which can be accomplished using a variety of purification protocols. Having separated the superantigen or the superantigen-targeting moiety conjugate from other proteins, the protein of interest may be further purified using chromatographic and electrophoretic techniques to achieve partial or complete purification (or purification to homogeneity). Analytical methods particularly suited to the preparation of a pure peptide are ion-exchange chromatography, size exclusion chromatography; affinity chromatography; polyacrylamide gel electrophoresis; isoelectric focusing. The term “purified” as used herein, is intended to refer to a composition, isolatable from other components, wherein the macromolecule (e.g., protein) of interest is purified to any degree relative to its original state. Generally, the terms “purified” refer to a macromolecule that has been subjected to fractionation to remove various other components, and which substantially retains its biological activity. The term “substantially purified” refers to a composition in which the macromolecule of interest forms the major component of the composition, such as constituting about 50%, about 60%, about 70%, about 80%, about 90%, about 95% or more of the content of the composition.

Various methods for quantifying the degree of purification of the protein are known to those of skill in the art, including, for example, determining the specific activity of an active fraction, and assessing the amount of a given protein within a fraction by SDS-PAGE analysis, High Performance Liquid Chromatography (HPLC), or any other fractionation technique. Various techniques suitable for use in protein purification include, for example, precipitation with ammonium sulfate, PEG, antibodies and the like or by heat denaturation, followed by centrifugation; chromatography steps such as ion exchange, gel filtration, reverse phase, hydroxyapatite, affinity chromatography; isoelectric focusing; gel electrophoresis; and combinations of such and other techniques. It is contemplated that the order of conducting the various purification steps may be changed, or that certain steps may be omitted, and still result in a suitable method for the preparation of a substantially purified protein or peptide.

### III. Immunopotentiator

It is contemplated that, in certain embodiments, the efficacy of the superantigen conjugate can be enhanced by administering the superantigen conjugate to the subject to be treated together with an immunopotentiator.

In certain embodiments, exemplary immunopotentiators can: (a) stimulate activating T-cell signaling, (b) repress T-cell inhibitory signalling between the cancerous cells and a T-cell, (c) repress inhibitory signalling that leads to T-cell expansion, activation and/or activity via a non-human IgG1-mediated immune response pathway, for example, a human IgG4 immunoglobulin-mediated pathway, (d) a combination of (a) and (b), (e) combination of (a) and (c), (f) a combination of (b) and (c), and (g) a combination of (a), (b), and (c).

In certain embodiments, the immunopotentiator is a checkpoint pathway inhibitor. A number of T-cell checkpoint inhibitor pathways have been identified to date, for example, the PD-1 immune checkpoint pathway and Cytotoxic T-lymphocyte antigen-4 (CTLA-4) immune checkpoint pathway.

PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, PD-L2, etc., that interact with PD-1 on the surface of T-cells to shut down or modulate T-cell activity. Using this approach, cancer can evade the T-cell mediated immune response.

In the CTLA-4 pathway, the interaction of CTLA-4 on the T-cell with its ligands (e.g., CD80, also known as B7-1, and CD86) on the surface of an antigen presenting cells (rather than the cancer calls) leads to T-cell inhibition. As a result, the ligand that inhibits T-cell activation or activity (e.g., CD80 or CD86) is provided by an antigen presenting cell (a key cell type in the immune system) rather than the cancer cell. Although CTLA-4 and PD-1 binding both have similar negative effects on T-cells the timing of downregulation, the responsible signaling mechanisms, and the anatomic locations of immune inhibition by these two immune checkpoints differ (American Journal of Clinical Oncology. Volume 39, Number 1, February 2016). Unlike CTLA-4, which is confined to the early priming phase of T-cell activation, PD-1 functions much later during the effector phase, (Keir et al. (2008) ANNU. REV IMMUNOL., 26:677-704). CTLA-4 and PD-1 represent two T-cell-inhibitory receptors with independent, non-redundant mechanisms of action.

In certain embodiments, the immunopotentiator prevents (completely or partially) an antigen expressed by the cancerous cell from repressing T-cell inhibitory signaling between the cancerous cell and the T-cell. In one embodiment, such an immunopotentiator is a checkpoint inhibitor, for example, a PD-1-based inhibitor. Examples of such immunopotentiators include, for example, anti-PD-1 antibodies, anti-PD-L1 antibodies, and anti-PD-L2 antibodies.

In certain embodiments, the superantigen conjugate is administered with a PD-1-based inhibitor. A PD-1-based inhibitor can include (i) a PD-1 inhibitor, i.e., a molecule (for example, an antibody or small molecule) that binds to PD-1 on a T-cell to prevent the binding of a PD-1 ligand expressed by the cancer cell of interest, and/or (ii) a PD-L inhibitor, e.g., a PD-L1 or PD-L2 inhibitor, i.e., a molecule (for example, an antibody or small molecule) that binds to a PD-1 ligand (for example, PD-L1 or PD-L2) to prevent the PD-1 ligand from binding to its cognate PD-1 on the T-cell.

In certain embodiments the superantigen conjugate is administered with a CTLA-4 inhibitor, e.g., an anti-CTLA-4 antibody. Exemplary anti-CTLA-4 antibodies include ipilimumab and tremelimumab.

In certain embodiments, the immunopotentiator prevents (completely or partially) an antigen expressed by the cancerous cell from repressing T-cell expansion, activation and/or activity via a human IgG4 (a non-human IgG1) mediated immune response pathway, for example, not via an ADCC pathway. It is contemplated that, in such embodiments, although the immune response potentiated by the superantigen conjugate and the immunopotentiator may include some ADCC activity, the principal component(s) of the immune response do not involve ADCC activity. In contrast, some of the antibodies currently being used in immunotherapy, such as ipilimumab (an anti-CTLA-4 IgG1 monoclonal antibody), can kill targeted cells via ADCC through signaling via their Fc domain through Fc receptors on effector cells. Ipilimumab, like many other therapeutic antibodies, was designed as a human IgG1 immunoglobulin, and although ipilimumab blocks interactions between CTLA-4 and CD80 or CD86, its mechanism of action is believed to include ADCC depletion of tumor-infiltrating regulatory T-cells that express high levels of cell surface CTLA-4. (Mahoney et al. (2015) NATURE REVIEWS, DRUG DISCOVERY 14: 561-584.) Given that CTLA-4 is highly expressed on a subset of T-cells (regulatory T-cells) that act to negatively control T-cells activation, when an anti-CTLA-4 IgG1 antibody is administered, the number of regulatory T-cells is reduced via ADCC.

In certain embodiments, it is desirable to use immunopotentiators whose mode of action is primarily to block the inhibitory signals between the cancer cells and the T-cells without significantly depleting the T-cell populations (for example, permitting the T-cell populations to expand). To achieve this, it is desirable to use an antibody, for example, an anti-PD-1 antibody, an anti-PD-L1 antibody or an anti-PD-L2 antibody, that has or is based on a human IgG4 isotype. Human IgG4 isotype is preferred under certain circumstances because this antibody isotype invokes little or no ADCC activity compared to the human IgG1 isotype (Mahoney et al. (2015) supra). Accordingly, in certain embodiments, the immunopotentiator, e.g., the anti-PD-1 antibody, anti-PD-L1 antibody, or anti-PD-L2 antibody has or is based on a human IgG4 isotype. In certain embodiments, the immunopotentiator is an antibody not known to deplete Tregs, e.g., IgG4 antibodies directed at non-CTLA-4 checkpoints (for example, anti-PD-1 IgG4 inhibitors).

In certain embodiments, the immunpotentiator is an antibody that has or is based on a human IgG1 isotype or another isotype that elicits antibody-dependent cell-mediated cytotoxicity (ADCC) and/or complement mediated cytotoxicity (CDC). In other embodiments, the immunpotentiator is an antibody that has or is based on a human IgG4 isotype or another isotype that elicits little or no antibody-dependent cell-mediated cytotoxicity (ADCC) and/or complement mediated cytotoxicity (CDC).

Exemplary PD-1-based inhibitors are described in U.S. Pat. Nos. 8,728,474, 8,952,136, and 9,073,994, and EP Patent No. 1537878B1. Exemplary anti-PD-1 antibodies include nivolumab (OPDIVO®, Bristol-Myers Squibb), pembrolizumab (KEYTRUDA®, Merck), cemiplimab (LIBTAYO®, Regeneron/Sanofi), spartalizumab (PDR001), MEDI0680 (AMP-514), pidilizumab (CT-011), dostarlimab, sintilimab, toripalimab, camrelizumab, tislelizumab, and prolgolimab. Exemplary anti-PD-L1 antibodies include avelumab (BAVENCIO®, EMD Serono/Pfizer), atezolizumab (TECENTRIQ®, Genentech), and durvalumab (IMFINZI®, Medimmune/AstraZeneca).

The PD-1-based inhibitor may be designed, expressed, and purified using techniques known to those skilled in the art, for example, as described hereinabove. The anti-PD-1 antibodies may be designed, expressed, purified, formulated and administered as described in U.S. Pat. Nos. 8,728,474, 8,952,136, and 9,073,994.

Other immunopotentiators (for example, antibodies, and various small molecules) may target signaling pathways involving one or more of the following ligands: B7-H3 (found on prostrate, renal cell, non-small cell lung, pancreatic, gastric, ovarian, colorectal cells, among others); B7-H4 (found on breast, renal cell, ovarian, pancreatic, melanoma cells, among others); HHLA2 (found on breast, lung, thyroid, melanoma, pancreas, ovary, liver, bladder, colon, prostate, kidney cells, among others); galectins (found on non-small cell lung, colorectal, and gastric cells, among others); CD30 (found on Hodgkin lymphoma, large cell lymphoma cells, among others); CD70 (found on non-Hodgkin's lymphoma, renal cells, among others); ICOSL (found on glioblastoma, melanoma cells, among others); CD155 (found on kidney, prostrate, pancreatic glioblastoma cells, among others); and TIM3. Similarly, other potential immunopotentiators that can be used include, for example, a 4-1BB (CD137) agonist (e.g., the fully human IgG4 anti-CD137 antibody Urelumab/BMS-663513), a LAG3 inhibitor (e.g., the humanized IgG4 anti-LAG3 antibody LAG525, Novartis); an IDO inhibitor (e.g., the small molecule INCB024360, Incyte Corporation), a TGFβ inhibitor (e.g., the small molecule Galunisertib, Eli Lilly) and other receptor or ligands that are found on T-cells and/or tumor cells. In certain embodiments, immunopotentiators (for example, antibodies, and various small molecules) that target signaling pathways involving one or more of the foregoing ligands are amenable to pharmaceutical intervention based on agonist/antagonist interactions but not through ADCC.

A. Antibody Production

Methods for producing antibodies are known in the art. For example, DNA molecules encoding light chain variable regions and heavy chain variable regions can be chemically synthesized using the sequences of the CDRs and variable regions of the antibodies of interest, for example, the antibody sequences provided in U.S. Pat. No. 8,952,136 and the hybridoma deposits described in U.S. Pat. No. 9,073,994. Synthetic DNA molecules can be ligated to other appropriate nucleotide sequences, including, e.g., constant region coding sequences, and expression control sequences, to produce conventional gene expression constructs encoding the desired antibodies. Production of defined gene constructs is within routine skill in the art. Alternatively, the sequences provided herein can be cloned out of hybridomas by conventional hybridization techniques or polymerase chain reaction (PCR) techniques, using synthetic nucleic acid probes whose sequences are based on sequence information provided herein, or prior art sequence information regarding genes encoding the heavy and light chains of murine antibodies in hybridoma cells.

Nucleic acids encoding the antibodies disclosed herein can be incorporated (ligated) into expression vectors, which can be introduced into host cells through conventional transfection or transformation techniques. Exemplary host cells are E. coli cells, Chinese hamster ovary (CHO) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art.

If a DNA construct encoding an antibody disclosed herein is to be expressed in eukaryotic host cells, e.g., CHO cells, it is first inserted into an expression vector containing a suitable eukaryotic promoter, a secretion signal, IgG enhancers, and various introns. This expression vector optionally contains sequences encoding all or part of a constant region, enabling an entire, or a part of, a heavy and/or light chain to be expressed. In some embodiments, a single expression vector contains both heavy and light chain variable regions to be expressed.

The gene construct can be introduced into eukaryotic host cells using conventional techniques. The host cells express V_{L} or V_{H} fragments, V_{L}-V_{H} heterodimers, V_{H}-V_{L} or V_{L}-V_{H} single chain polypeptides, complete heavy or light immunoglobulin chains, or portions thereof, each of which may be attached to a moiety having another function (e.g., cytotoxicity). In some embodiments, a host cell is transfected with a single vector expressing a polypeptide expressing an entire, or part of, a heavy chain (e.g., a heavy chain variable region) or a light chain (e.g., a light chain variable region). In other embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain. In still other embodiments, a host cell is co-transfected with more than one expression vector (e.g., one expression vector expressing a polypeptide comprising an entire, or part of, a heavy chain or heavy chain variable region, and another expression vector expressing a polypeptide comprising an entire, or part of, a light chain or light chain variable region).

A method of producing a polypeptide comprising an immunoglobulin heavy chain variable region or a polypeptide comprising an immunoglobulin light chain variable region may comprise growing (culturing) a host cell transfected with an expression vector under conditions that permits expression of the polypeptide comprising the immunoglobulin heavy chain variable region or the polypeptide comprising the immunoglobulin light chain variable region. The polypeptide comprising a heavy chain variable region or the polypeptide comprising the light chain variable region then may be purified using techniques well known in the art, e.g., affinity tags such as glutathione-S-transferase (GST) and histidine tags.

A method of producing a monoclonal antibody that binds a target protein, for example, PD-1, PD-L1, or PD-L2, or an antigen-binding fragment of the antibody, may comprise growing a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial chains), under conditions that permit expression of both chains. The intact antibody (or antigen-binding fragment) can be harvested and purified using techniques well known in the art, e.g., Protein A, Protein G, affinity tags such as glutathione-S-transferase (GST) and histidine tags. It is within ordinary skill in the art to express the heavy chain and the light chain from a single expression vector or from two separate expression vectors.

B. Antibody Modifications

Methods for reducing or eliminating the antigenicity of antibodies and antibody fragments are known in the art. When the antibodies are to be administered to a human, the antibodies preferably are “humanized” to reduce or eliminate antigenicity in humans. Preferably, a humanized antibody has the same or substantially the same affinity for the antigen as the non-humanized mouse antibody from which it was derived.

In one humanization approach, chimeric proteins are created in which mouse immunoglobulin constant regions are replaced with human immunoglobulin constant regions. See, e.g., Morrison et al. (1984) PROC. NAT. ACAD. SCI. 81:6851-6855, Neuberger et al. (1984) NATURE 312:604-608; U.S. Pat. No. 6,893,625 (Robinson); U.S. Pat. No. 5,500,362 (Robinson); and 4,816,567 (Cabilly).

In an approach known as CDR grafting, the CDRs of the light and heavy chain variable regions are grafted into frameworks from another species. For example, murine CDRs can be grafted into human FRs. In some embodiments, the CDRs of the light and heavy chain variable regions of an anti-ErbB3 antibody are grafted onto human FRs or consensus human FRs. To create consensus human FRs, FRs from several human heavy chain or light chain amino acid sequences are aligned to identify a consensus amino acid sequence. CDR grafting is described in U.S. Pat. No. 7,022,500 (Queen); U.S. Pat. No. 6,982,321 (Winter); U.S. Pat. No. 6,180,370 (Queen); U.S. Pat. No. 6,054,297 (Carter); U.S. Pat. No. 5,693,762 (Queen); U.S. Pat. No. 5,859,205 (Adair); U.S. Pat. No. 5,693,761 (Queen); U.S. Pat. No. 5,565,332 (Hoogenboom); U.S. Pat. No. 5,585,089 (Queen); U.S. Pat. No. 5,530,101 (Queen); Jones et al. (1986) NATURE 321: 522-525; Riechmann et al. (1988) NATURE 332: 323-327; Verhoeyen et al. (1988) SCIENCE 239: 1534-1536; and Winter (1998) FEBS LETT 430: 92-94.

In an approach called “SUPERHUMANIZATION™,” human CDR sequences are chosen from human germline genes, based on the structural similarity of the human CDRs to those of the mouse antibody to be humanized See, e.g., U.S. Pat. No. 6,881,557 (Foote); and Tan et al. (2002) J. IMMUNOL. 169:1119-1125.

Other methods to reduce immunogenicity include “reshaping,” “hyperchimerization,” and “veneering/resurfacing.” See, e.g., Vaswami et al. (1998) ANNALS OF ALLERGY, ASTHMA, & IMMUNOL. 81:105; Roguska et al. (1996) PROT. ENGINEER 9:895-904; and U.S. Pat. No. 6,072,035 (Hardman) In the veneering/resurfacing approach, the surface accessible amino acid residues in the murine antibody are replaced by amino acid residues more frequently found at the same positions in a human antibody. This type of antibody resurfacing is described, e.g., in U.S. Pat. No. 5,639,641 (Pedersen).

Another approach for converting a mouse antibody into a form suitable for medical use in humans is known as ACTIVMAB™ technology (Vaccinex, Inc., Rochester, N.Y.), which involves a vaccinia virus-based vector to express antibodies in mammalian cells. High levels of combinatorial diversity of IgG heavy and light chains are said to be produced. See, e.g., U.S. Pat. No. 6,706,477 (Zauderer); U.S. Pat. No. 6,800,442 (Zauderer); and 6,872,518 (Zauderer).

Another approach for converting a mouse antibody into a form suitable for use in humans is technology practiced commercially by KaloBios Pharmaceuticals, Inc. (Palo Alto, Calif.). This technology involves the use of a proprietary human “acceptor” library to produce an “epitope focused” library for antibody selection.

Another approach for modifying a mouse antibody into a form suitable for medical use in humans is HUMAN ENGINEERING™ technology, which is practiced commercially by XOMA (US) LLC. See, e.g., PCT Publication No. WO 93/11794 and U.S. Pat. Nos. 5,766,886; 5,770,196; 5,821,123; and 5,869,619.

Any suitable approach, including any of the above approaches, can be used to reduce or eliminate human immunogenicity of an antibody including the binding moiety component of the superantigen conjugate disclosed herein.

Methods of making multispecific antibodies are known in the art. Multi-specific antibodies include bispecific antibodies. Bispecific antibodies are antibodies that have binding specificities for at least two different epitopes. Exemplary bispecific antibodies bind to two different epitopes of the antigen of interest. Bispecific antibodies can be prepared as full length antibodies or antibody fragments (e.g., F(ab′)2 bispecific antibodies and diabodies) as described, for example, in Milstein et al., NATURE 305:537-539 (1983), WO 93/08829, Traunecker et al., EMBO J., 10:3655-3659 (1991), WO 94/04690, Suresh et al. (1986) METHODS IN ENZYMOLOGY 121:210, WO96/27011, Brennan et al. (1985) SCIENCE 229: 81, Shalaby et al. (1992) J. EXP. MED. 175: 217-225, Kostelny et al. (1992) J. IMMUNOL. 148(5):1547-1553, Hollinger et al. (1993) PNAS, 90:6444-6448, Gruber et al. (1994) J. IMMUNOL. 152:5368, Wu et al. (2007) NAT. BIOTECHNOL. 25(11): 1290-1297, U.S. Patent Publication No. 2007/0071675, and Bostrom et al., SCIENCE 323:1640-1644 (2009).

### IV. Formulations and Pharmaceutical Compositions

The superantigen conjugate and optional immunopotentiator, e.g., PD-1-based inhibitor, can be administered to the subject so as to treat the cancer, for example, to slow the growth rate of cancer cells, reduce the incidence or number of metastases, reduce tumor size, inhibit tumor growth, reduce the blood supply to a tumor or cancer cells, promote an immune response against cancer cells or a tumor, prevent or inhibit the progression of cancer, for example, by at least 40%, 50%, 60%, 70%, 80%, 90%, 95%, 98%, 99%, or 100%. Alternatively, the superantigen conjugate and optional immunopotentiator, e.g., PD-1-based inhibitor, can be administered to the subject so as to treat the cancer, for example, to increase the lifespan of a subject with cancer, for example, by 3 months, 6 months, 9 months, 12 months, 1 year, 2 years, 3 years, 4, years, 5 years, 6 years, 7 years, 8 years, 9 years, or 10 years. Alternatively, the superantigen conjugate and optional immunopotentiator, e.g., PD-1-based inhibitor, can be administered to the subject so as to treat the cancer, for example, to facilitate cancer free survival of a subject following cancer treatment, for example, for 3 months, 6 months, 9 months, 12 months, 1 year, 2 years, 3 years, 4 years, 5 years, 6 years, 7 years, 8 years, 9 years, or 10 years. Alternatively, the superantigen conjugate and optional immunopotentiator, e.g., PD-1-based inhibitor, can be administered to the subject so as to treat the cancer, for example, to prevent cancer progression in a subject following cancer treatment, for example, for 3 months, 6 months, 9 months, 12 months, 1 year, 2 years, 3 years, 4 years, 5 years, 6 years, 7 years, 8 years, 9 years, or 10 years. In each approach, the superantigen conjugate and immunopotentiator, e.g., PD-1-based inhibitor, can be administered together, sequentially, or intermittently to the subject

The superantigen conjugate and the immunopotentiator, e.g., PD-1-based inhibitor, may be formulated separately or together using techniques known to those skilled in the art. For example, for therapeutic use, the superantigen conjugate and/or the immunopotentiator, e.g., PD-1-based inhibitor, is combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Pharmaceutical compositions containing the superantigen and/or the immunopotentiator, e.g., PD-1-based inhibitor, disclosed herein can be provided in a single dosage form or different dosage forms. The pharmaceutical composition or compositions should be formulated to be compatible with its intended route of administration. Examples of routes of administration are intravenous (IV), intramuscular, intradermal, inhalation, transdermal, topical, transmucosal, and rectal administration. Alternatively, the agents may be administered locally rather than systemically, for example, via injection of the agent or agents directly into the site of action, often in a depot or sustained release formulation.

Useful formulations can be prepared by methods well known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

The superantigen conjugate and/or immunopotentiator, e.g., PD-1-based inhibitor, of the present invention may be employed alone or in conjunction with other compounds, such as carriers or other therapeutic compounds. Pharmaceutical compositions of the present invention comprise an effective amount of one or more superantigen conjugates and optionally one or more immunopotentiators, for example a PD-1-based inhibitor, for example an anti-PD-1 antibody, and may also contain additional agents, dissolved or dispersed in a pharmaceutically acceptable carrier. The phrases “pharmaceutical” or “pharmacologically acceptable” refer to substances, e.g., compositions, that do not produce an adverse, allergic or other untoward reaction when administered to a mammal, such as, for example, a human. The preparation of a pharmaceutical composition that contains at least one superantigen conjugate and/or immunopotentiator, e.g., PD-1-based inhibitor, will be known to those of skill in the art in light of the present disclosure, and as exemplified by Remington's Pharmaceutical Sciences, 18th Ed. Mack Printing Company, 1990, incorporated herein by reference. Moreover, for human administration, it will be understood that preparations should meet sterility, pyrogenicity, general safety and purity standards as required by FDA Office of Biological Standards.

In a specific embodiment of the invention, the compositions of the invention comprise tumor-targeted superantigen in combination with a PD-1-based inhibitor. Such combinations include, for example, any tumor-targeted superantigen and/or PD-1-based inhibitor as described herein.

In a specific embodiment of the invention, the tumor-targeted super antigen comprises a bacterial superantigen including, but are not limited to, Staphylococcal enterotoxin (SE), Streptococcus pyogenes exotoxin (SPE), Staphylococcus aureus toxic shock-syndrome toxin (TSST-1), Streptococcal mitogenic exotoxin (SME), Streptococcal superantigen (SSA), Staphylococcal enterotoxin A (SEA), Staphylococcal enterotoxin B (SEB), and Staphylococcal enterotoxin E (SEE) conjugated to a targeting moiety. In another embodiment of the invention, the compositions comprise tumor-targeted superantigens comprising superantigens with the following Protein Data Bank and/or GenBank accession numbers include, but are not limited to, SEE is P12993; SEA is P013163; SEB is P01552; SECT is P01553; SED is P20723; and SEH is AAA19777, as well as variants thereof, conjugated to a targeting moiety.

In certain embodiments, the superantigen conjugate comprises a wild type or engineered superantigen sequence such as, the wild-type SEE sequence (SEQ ID NO: 1) or the wild type SEA sequence (SEQ ID NO: 2), either of which can be modified such that amino acids in any of the identified regions A-E (see, FIG. 2) are substituted with other amino acids. In certain embodiments, the superantigen incorporated in the conjugate is SEA/E-120 (SEQ ID NO: 3) or SEA_{D227A} (SEQ ID NO: 4).

Specific examples of targeting moieties to be conjugated to the superantigens include, for example, any molecule that is able to bind to a cellular molecule and preferably a disease specific molecule such as a cancer cell specific molecule. The targeting moiety can be selected from antibodies, including antigen binding fragments, soluble T-cell receptors, growth factors, interleukins, hormones, etc. Exemplary cancer targeting antibodies can include, but are not limited to, anti-CD19, anti-CD20 antibodies, anti-5T4 antibodies, anti-Ep-CAM antibodies, anti-Her-2/neu antibodies, anti-EGFR antibodies, anti-CEA antibodies, anti-prostate specific membrane antigen (PSMA) antibodies, and anti-IGF-1R antibodies. In one embodiment, the superantigen can be conjugated to an immunologically reactive antibody fragment such as C215Fab, 5T4Fab (see, WO8907947) or C242Fab (see, WO9301303).

Examples of such tumor-targeted superantigens include C215Fab-SEA (SEQ ID NO: 5), 5T4Fab-SEA_{D227A} (SEQ ID NO: 6) and 5T4Fab-SEA/E-120 (SEQ ID NO: 7). In a preferred embodiment, the superantigen conjugate is 5T4 Fab-SEA/E-120, known in the art as naptumomab estafenatox/ANYARA®, which comprises two polypeptide sequences that together define an Fab fragment of an anti-5T4 antibody, where one of the polypeptide sequences further comprises the SEA/E-120 superantigen namely SEQ ID NO: 8 (chimeric V_{H} chain of 5T4 Fab coupled by three amino acid linker to SEA/E-120) and SEQ ID NO: 9 (chimeric V_{L} chain of 5T4 Fab).

In a preferred embodiment, the compositions of the invention comprise the tumor-targeted superantigen 5T4Fab-SEA/E-120, known in the art as naptumomab estafenatox/ANYARA® optionally in combination with a PD-1-based inhibitor, such as an anti-PD-1 antibody, for example, nivolumab (OPDIVO®, Bristol-Myers Squibb), pembrolizumab (KEYTRUDA®, Merck), cemiplimab (LIBTAYO®, Regeneron/Sanofi), spartalizumab (PDR001), MEDI0680 (AMP-514), pidilizumab (CT-011), dostarlimab, sintilimab, toripalimab, camrelizumab, tislelizumab, and prolgolimab, or an anti-PD-L1 antibody such as avelumab (BAVENCIO®, EMD Serono/Pfizer), atezolizumab (TECENTRIQ®, Genentech), and durvalumab (IMFINZI®, Medimmune/AstraZeneca).

Formulations or dosage form containing the superantigen conjugate and optional immunopotentiator, e.g., PD-1-based inhibitor, may comprise different types of carriers depending on whether they are to be administered in solid, liquid or aerosol form, and whether it need to be sterile for such routes of administration as injection.

Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In certain embodiments, pharmaceutical compositions may comprise, for example, at least about 0.1% of an active compound. In other embodiments, the active compound may comprise between about 2% to about 75% of the weight of the unit, or between about 25% to about 60%, for example, and any range derivable therein. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable. Such determinations are known and used by those of skill in the art.

The active agents are administered in an amount or amounts effective to decrease, reduce, inhibit or otherwise abrogate the growth or proliferation of cancer cells, induce apoptosis, inhibit angiogenesis of a cancer or tumor, inhibit metastasis, or induce cytotoxicity in cells. The effective amount of active compound(s) used to practice the present invention for therapeutic treatment of cancer varies depending upon the manner of administration, the age, body weight, and general health of the subject. These terms include synergistic situations such as those presented and described in the instant invention wherein a single agent alone, such as a superantigen conjugate or a immunopotentiator, e.g., PD-1-based inhibitor, may act weakly or not at all, but when combined with each other, for example, but not limited to, via sequential dosage, the two or more agents act to produce a synergistic result.

In certain non-limiting examples, a dose of the superantigen conjugate may also comprise from about 1 microgram/kg/body weight, about 5 microgram/kg/body weight, about 10 microgram/kg/body weight, about 15 microgram/kg/body weight, about 20 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, about 1 microgram/kg/body weight to about 100 milligram/kg/body weight. Other exemplary dosage ranges, range from about 1 microgram/kg/body weight to about 1000 microgram/kg/body weight, from about 1 microgram/kg/body weight to about 100 microgram/kg/body weight, from about 1 microgram/kg/body weight to about 75 microgram/kg/body weight, from about 1 microgram/kg/body weight to about 50 microgram/kg/body weight, from about 1 microgram/kg/body weight to about 40 microgram/kg/body weight, from about 1 microgram/kg/body weight to about 30 microgram/kg/body weight, from about 1 microgram/kg/body weight to about 20 microgram/kg/body weight, from about 1 microgram/kg/body weight to about 15 microgram/kg/body weight, from about 1 microgram/kg/body weight to about 10 microgram/kg/body weight, from about 5 microgram/kg/body weight to about 1000 microgram/kg/body weight, from about 5 microgram/kg/body weight to about 100 microgram/kg/body weight, from about 5 microgram/kg/body weight to about 75 microgram/kg/body weight, from about 5 microgram/kg/body weight to about 50 microgram/kg/body weight, from about 5 microgram/kg/body weight to about 40 microgram/kg/body weight, from about 5 microgram/kg/body weight to about 30 microgram/kg/body weight, from about 5 microgram/kg/body weight to about 20 microgram/kg/body weight, from about 5 microgram/kg/body weight to about 15 microgram/kg/body weight, from about 5 microgram/kg/body weight to about 10 microgram/kg/body weight, from about 10 microgram/kg/body weight to about 1000 microgram/kg/body weight, from about 10 microgram/kg/body weight to about 100 microgram/kg/body weight, from about 10 microgram/kg/body weight to about 75 microgram/kg/body weight, from about 10 microgram/kg/body weight to about 50 microgram/kg/body weight, from about 10 microgram/kg/body weight to about 40 microgram/kg/body weight, from about 10 microgram/kg/body weight to about 30 microgram/kg/body weight, from about 10 microgram/kg/body weight to about 20 microgram/kg/body weight, from about 10 microgram/kg/body weight to about 15 microgram/kg/body weight, from about 15 microgram/kg/body weight to about 1000 microgram/kg/body weight, from about 15 microgram/kg/body weight to about 100 microgram/kg/body weight, from about 15 microgram/kg/body weight to about 75 microgram/kg/body weight, from about 15 microgram/kg/body weight to about 50 microgram/kg/body weight, from about 15 microgram/kg/body weight to about 40 microgram/kg/body weight, from about 15 microgram/kg/body weight to about 30 microgram/kg/body weight, from about 15 microgram/kg/body weight to about 20 microgram/kg/body weight, from about 20 microgram/kg/body weight to about 1000 microgram/kg/body weight, from about 20 microgram/kg/body weight to about 100 microgram/kg/body weight, from about 20 microgram/kg/body weight to about 75 microgram/kg/body weight, from about 20 microgram/kg/body weight to about 50 microgram/kg/body weight, from about 20 microgram/kg/body weight to about 40 microgram/kg/body weight, from about 20 microgram/kg/body weight to about 30 microgram/kg/body weight, etc., can be administered, based on the numbers described above.

In certain embodiments, for example, but not limited to, administration of the superantigen conjugate, the effective amount or dose of the superantigen conjugate that is administered is an amount in the range of 0.01 to 500 μg/kg body weight of the subject, for example, 0.1-500 μg/kg body weight of the subject, and, for example, 1-100 μg/kg body weight of the subject.

It is envisioned that the effective amount or dose of the immunopotentiator, e.g., PD-1-based inhibitor, that is administered in combination with the superantigen conjugate is a dose that results in an at least an additive but preferably a synergistic anti-tumor effect and does not interfere or inhibit the enhancement of the immune system or T-cell activation. If the immunopotentiator, e.g., PD-1-based inhibitor, is administered simultaneously with the superantigen conjugate, then the immunopotentiator may be administered in a low dose such that it does not interfere with the mechanism of action of the superantigen conjugate.

Generally, a therapeutically effective amount of PD-1-based inhibitor, for example, the anti-PD-1 antibody, is in the range of 0.1 mg/kg to 100 mg/kg, e.g., 1 mg/kg to 100 mg/kg, 1 mg/kg to 10 mg/kg. For example, pembrolizumab (KEYTRUDA®) can be administered periodically at 2 mg/kg as an intravenous infusion. The amount of PD-based inhibitor administered will depend on variables such as the type and extent of disease or indication to be treated, the overall health of the patient, the in vivo potency of the superantigen conjugate and the PD-1-based inhibitor, the pharmaceutical formulation, and the route of administration.

### V. Treatment Regimens and Indications

Treatment regimens may vary as well, and often depend on tumor type, tumor location, disease progression, and health and age of the patient. Certain types of tumor may require more aggressive treatment protocols, but at the same time, the patients may be unable to tolerate more aggressive treatment regimens. The clinician may often be best suited to make such decisions based on his or her skill in the art and the known efficacy and toxicity (if any) of the therapeutic formulations.

In a specific embodiment of the invention, the treatment methods of the invention comprise administration of a tumor-targeted superantigen, optionally in combination with an immunopotentiator, e.g., PD-1-based inhibitor, to a patient in need thereof, i.e., a cancer patient. Such combination treatments include, for example, administration of any tumor-targeted superantigen and/or immunopotentiator, e.g., PD-1-based inhibitor, as described herein. In a specific embodiment of the invention, the tumor-targeted super antigen comprises a bacterial superantigen including, but are not limited to, Staphylococcal enterotoxin (SE), Streptococcus pyogenes exotoxin (SPE), Staphylococcus aureus toxic shock-syndrome toxin (TSST-1), Streptococcal mitogenic exotoxin (SME), Streptococcal superantigen (SSA), Staphylococcal enterotoxin A (SEA), Staphylococcal enterotoxin B (SEB), and Staphylococcal enterotoxin E (SEE) conjugated to a targeting moiety.

In certain embodiments, the superantigen conjugate comprises a wild type or engineered superantigen sequence such as, the wild-type SEE sequence (SEQ ID NO: 1) or the wild type SEA sequence (SEQ ID NO: 2), either of which can be modified such that amino acids in any of the identified regions A-E (see, FIG. 2) are substituted with other amino acids. In certain embodiments, the superantigen incorporated in the conjugate is SEA/E-120 (SEQ ID NO: 3) or SEA_{D227A} (SEQ ID NO: 4).

Specific examples of targeting moieties to be conjugated to the superantigens include, for example, any molecule that is able to bind to a cellular molecule and preferably a disease specific molecule such as a cancer cell specific molecule. The targeting moiety can be selected from antibodies, including antigen binding fragments, soluble T-cell receptors, growth factors, interleukins, hormones, etc. Exemplary cancer targeting antibodies can include, but are not limited to, anti-CD19, anti-CD20 antibodies, anti-5T4 antibodies, anti-Ep-CAM antibodies, anti-Her-2/neu antibodies, anti-EGFR antibodies, anti-CEA antibodies, anti-prostate specific membrane antigen (PSMA) antibodies, and anti-IGF-1R antibodies. In one embodiment, the superantigen can be conjugated to an immunologically reactive antibody fragment such as C215Fab, 5T4Fab (see, WO8907947) or C242Fab (see, WO9301303).

Examples of such tumor-targeted superantigens include C215Fab-SEA (SEQ ID NO: 5), 5T4Fab-SEA_{D227A} (SEQ ID NO: 6) and 5T4Fab-SEA/E-120 (SEQ ID NO: 7). In a preferred embodiment, the superantigen conjugate is 5T4 Fab-SEA/E-120 known in the art as naptumomab estafenatox/ANYARA®, which comprises two polypeptide sequences that together define an Fab fragment of an anti-5T4 antibody, where one of the polypeptide sequences further comprises the SEA/E-120 superantigen namely SEQ ID. NO: 8 (chimeric V_{H} chain of 5T4 Fab coupled by three amino acid linker to SEA/E-120) and SEQ ID. NO: 9 (chimeric V_{L} chain of 5T4 Fab).

In a preferred embodiment, the compositions of the invention comprise the tumor-targeted superantigen 5T4Fab-SEA/E-120, known in the art as naptumomab estafenatox/ANYARA® optionally in combination with a PD-1-based inhibitor, such as an anti-PD-1 antibody, for example, nivolumab (OPDIVO®, Bristol-Myers Squibb), pembrolizumab (KEYTRUDA®, Merck), cemiplimab (LIBTAYO®, Regeneron/Sanofi), spartalizumab (PDR001), MEDI0680 (AMP-514), pidilizumab (CT-011), dostarlimab, sintilimab, toripalimab, camrelizumab, tislelizumab, and prolgolimab, or an anti-PD-L1 antibody such as avelumab (BAVENCIO®, EMD Serono/Pfizer), atezolizumab (TECENTRIQ®, Genentech), and durvalumab (IMFINZI®, Medimmune/AstraZeneca).

Furthermore, the superantigen conjugate and/or immunopotentiator, e.g., PD-1-based inhibitor, may be co-administered together or sequentially with one or more additional agents that enhance the potency and/or selectively of the therapeutic effect. Such agents include, for example, corticosteroids, additional immune modulators, and those compounds designed to reduce the patient's possible immunoreactivity to the administered superantigen conjugate. For example, immunoreactivity to the administered superantigen may be reduced via co-administration with, for example, an anti-CD20 antibody and/or an anti-CD19 antibody, that reduces the production of anti-superantigen antibodies in the subject.

Preferably, patients to be treated will have adequate bone marrow function (defined as a peripheral absolute granulocyte count of >2,000/mm³ and a platelet count of 100,000/mm³), adequate liver function (bilirubin<1.5 mg/dl) and adequate renal function (creatinine<1.5 mg/dl).

In certain embodiments, the treatment regimen of the present invention may involve contacting the neoplasm or tumor cells with the superantigen conjugate and the immunopotentiator, e.g., PD-1-based inhibitor, at the same time. This may be achieved by contacting the cell with a single composition or pharmacological formulation that includes both agents, or by contacting the cell with two distinct compositions or formulations, at the same time, wherein one composition includes the superantigen conjugate and the other includes the immunopotentiator, e.g., PD-1-based inhibitor.

Alternatively, the superantigen conjugate may precede or follow the immunopotentiator, e.g., PD-1-based inhibitor, inhibitor by intervals ranging from minutes, days to weeks. In embodiments where immunopotentiator and the superantigen conjugate are applied separately to the cell, one should ensure that a significant period of time does not expire between the time of each delivery, such that the superantigen conjugate and immunopotentiator would still be able to exert an advantageously combined effect on the cell. In such instances, it is contemplated that one may contact the cell with both modalities within about 12-72 hours of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, the superantigen conjugate being “A” and the immunopotentiator, e.g., PD-1-based inhibitor, being “B”: A/B/A, B/A/B, B/B/A, A/A/B, A/B/B, B/A/A, A/B/B/B, B/A/B/B, B/B/B/A, B/B/A/B, A/A/B/B, A/B/A/B, A/B/B/A, B/B/A/A, B/A/B/A, B/A/A/B, A/A/A/B, B/A/A/A, A/B/A/A, and A/A/B/A.

It is further envisioned that the present invention can be used in combination with surgical intervention. In the case of surgical intervention, the present invention may be used preoperatively, e.g., to render an inoperable tumor subject to resection. Alternatively, the present invention may be used at the time of surgery, and/or thereafter, to treat residual or metastatic disease. For example, a resected tumor bed may be injected or perfused with a formulation comprising the tumor-targeted superantigen and/or the immunopotentiator, e.g., PD-1-based inhibitor. The perfusion may be continued post-resection, for example, by leaving a catheter implanted at the site of the surgery. Periodic post-surgical treatment also is envisioned. Any combination of the invention therapy with surgery is within the scope of the invention.

Continuous administration also may be applied where appropriate, for example, where a tumor is excised and the tumor bed is treated to eliminate residual, microscopic disease. Delivery via syringe or cauterization is preferred. Such continuous perfusion may take place for a period from about 1-2 hours, to about 2-6 hours, to about 6-12 hours, to about 12-24 hours, to about 1-2 days, to about 1-2 weeks or longer following the initiation of treatment. Generally, the dose of the therapeutic composition via continuous perfusion will be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

A typical course of treatment, for a primary tumor or a post-excision tumor bed, may involve multiple doses. Typical primary tumor treatment may involve a 6 dose application over a two-week period. The two-week regimen may be repeated one, two, three, four, five, six or more times. During a course of treatment, the need to complete the planned dosings may be re-evaluated.

Immunotherapy with the superantigen conjugate often results in rapid (within hours) and powerful polyclonal activation of T lymphocytes. A superantigen conjugate treatment cycle may include 4 to 5 daily intravenous superantigen conjugate drug injections. Such treatment cycles can be given in e.g., 4 to 6 week intervals. The inflammation with infiltration of CTLs into the tumor is one of the major effectors of the anti-tumor therapeutic superantigens. After a short period of massive activation and differentiation of CTLs, the T-cell response declines rapidly (within 4-5 days) back to base line levels. Thus, the period of lymphocyte proliferation, during which cytostatic drugs may interfere with superantigen treatment is short and well-defined. Only with the superantigen/immunopotentiator therapy of the instant invention is such a distinct time frame for activity plausible, thereby allowing the novel integrated high dose cytostatic agent/immunotherapy treatment.

In certain embodiments, a subject is administered a superantigen conjugate, e.g., a superantigen conjugate contemplated herein, daily for 2 to 6 consecutive days (e.g., 2, 3, 4, 5, or 6 consecutive days) every 2 to 12 weeks (e.g., 2, 3, 4, 5, 6, 7, 8, 9, 10, 11, or 12 weeks). In certain embodiments, a subject is administered a PD-1-based inhibitor, e.g., an anti-PD-1 antibody, e.g., an anti-PD-1 antibody contemplated herein, every 1 to 5 weeks (e.g., every 1, 2, 3, 4, or 5 weeks). In certain embodiments, a subject is administered (i) the superantigen conjugate daily for 2 to 6 consecutive days (e.g., 2, 3, 4, 5, or 6 consecutive days) every 2 to 12 weeks (e.g., 2, 3, 4, 5, 6, 7, 8, 9, 10, 11, or 12 weeks) and (ii) the PD-1-based inhibitor every 1 to 5 weeks (e.g., every 1, 2, 3, 4, or 5 weeks). In certain embodiments, a subject is administered (i) the superantigen conjugate daily for 4 consecutive days every 3 to 4 weeks (e.g., 3 or 4 weeks) and (ii) the PD-1-based inhibitor every 2 to 4 weeks (e.g., every 2, 3, or 4 weeks).

It is contemplated that a number of cancers may be treated using the methods and compositions described herein, including but not limited to primary or metastatic melanoma, adenocarcinoma, squamous cell carcinoma, adenosquamous cell carcinoma, thymoma, lymphoma, sarcoma, lung cancer, liver cancer, non-Hodgkin's lymphoma, Hodgkin's lymphoma, leukemia, uterine cancer, breast cancer, prostate cancer, ovarian cancer, pancreatic cancer, colon cancer, multiple myeloma, neuroblastoma, NPC, bladder cancer, cervical cancer and the like.

Moreover, the cancer that may be treated using the methods and compositions described herein may be based upon the body location and/or system to be treated, for example, but not limited to bone (e.g., Ewing's Family of tumors, osteosarcoma); brain (e.g., adult brain tumor, (e.g., adult brain tumor, brain stem glioma (childhood), cerebellar astrocytoma (childhood), cerebral astrocytoma/malignant glioma (childhood), ependymoma (childhood). medulloblastoma (childhood), supratentorial primitive neuroectodermal tumors and pineoblastoma (childhood), visual pathway and hypothalamic glioma (childhood) and childhood brain tumor (other)); breast (e.g., female or male breast cancer); digestive/gastrointestinal (e.g., anal cancer, bile duct cancer (extrahepatic), carcinoid tumor (gastrointestinal), colon cancer, esophageal cancer, gallbladder cancer, liver cancer (adult primary), liver cancer (childhood), pancreatic cancer, small intestine cancer, stomach (gastric) cancer); endocrine (e.g., adrenocortical carcinoma, carcinoid tumor (gastrointestinal), islet cell carcinoma (endocrine pancreas), parathyroid cancer, pheochromocytoma, pituitary tumor, thyroid cancer); eye (e.g., melanoma (intraocular), retinoblastoma); genitourinary (e.g., bladder cancer, kidney (renal cell) cancer, penile cancer, prostate cancer, renal pelvis and ureter cancer (transitional cell), testicular cancer, urethral cancer, Wilms' Tumor and other childhood kidney tumors); germ cell (e.g., extracranial germ cell tumor (childhood), extragonadal germ cell tumor, ovarian germ cell tumor, testicular cancer); gynecologic (e.g., cervical cancer, endometrial cancer, gestational trophoblastic tumor, ovarian epithelial cancer, ovarian germ cell tumor, ovarian low malignant potential tumor, uterine sarcoma, vaginal cancer, vulvar cancer); head and neck (e.g., hypopharyngeal cancer, laryngeal cancer, lip and oral cavity cancer, metastatic squamous neck cancer with occult primary, nasopharyngeal cancer, oropharyngeal cancer, paranasal sinus and nasal cavity cancer, parathyroid cancer, salivary gland cancer); lung (e.g., non-small cell lung cancer, small cell lung cancer); lymphoma (e.g., AIDS-Related Lymphoma, cutaneous T-cell lymphoma, Hodgkin's Lymphoma (adult), Hodgkin's Lymphoma (childhood), Hodgkin's Lymphoma during pregnancy, mycosis fungoides, Non-Hodgkin's Lymphoma (adult), Non-Hodgkin's Lymphoma (childhood), Non-Hodgkin's Lymphoma during pregnancy, primary central nervous system lymphoma, Sezary Syndrome, T-cell lymphoma (cutaneous), Waldenstrom's Macroglobulinemia); musculoskeletal (e.g., Ewing's Family of tumors, osteosarcoma/malignant fibrous histiocytoma of bone, rhabdomyosarcoma (childhood), soft tissue sarcoma (adult), soft tissue sarcoma (childhood), uterine sarcoma); neurologic (e.g., adult brain tumor, childhood brain tumor (e.g., brain stem glioma, cerebellar astrocytoma, cerebral astrocytoma/malignant glioma, ependymoma, medulloblastoma, supratentorial primitive neuroectodermal tumors and pineoblastoma, visual pathway and hypothalamic glioma, other brain tumors), neuroblastoma, pituitary tumor primary central nervous system lymphoma); respiratory/thoracic (e.g., non-small cell lung cancer, small cell lung cancer, malignant mesothelioma, thymoma and thymic carcinoma); and skin (e.g., cutaneous T-cell lymphoma, Kaposi's sarcoma, melanoma, and skin cancer).

It is understood that the method can be used to treat a variety of cancers, for example, a cancer selected from breast cancer, bladder cancer, cervical cancer, colon cancer, colorectal cancer, endometrial cancer, gastric cancer, head and neck cancer, liver cancer, melanoma, mesothelioma, non-small cell lung cancer, ovarian cancer, pancreatic cancer, prostate cancer, renal cell cancer, and skin cancer.

Yet further, the cancer may include a tumor comprised of tumor cells. For example, tumor cells may include, but are not limited to melanoma cell, a bladder cancer cell, a breast cancer cell, a lung cancer cell, a colon cancer cell, a prostate cancer cell, a liver cancer cell, a pancreatic cancer cell, a stomach cancer cell, a testicular cancer cell, a renal cancer cell, an ovarian cancer cell, a lymphatic cancer cell, a skin cancer cell, a brain cancer cell, a bone cancer cell, or a soft tissue cancer cell. Examples of solid tumors that can be treated according to the invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

### VI. Kits

In addition, the invention provides kits comprising, for example, a first container containing a superantigen conjugate and a second container containing an immunopotentiator, e.g., a PD-1-based inhibitor, such as an anti-PD-1 antibody. Such a kit may also contain additional agents such as, for example, corticosteroid or another lipid modulator. The container means may itself be a syringe, pipette, and/or other such like apparatus, from which the formulation may be applied to a specific area of the body, injected into an animal, and/or applied and/or mixed with the other components of the kit.

The kits may comprise a suitably aliquoted superantigen conjugate and/or immunopotentiator, e.g., PD-1-based inhibitor, and optionally, lipid and/or additional agent compositions of the present invention. The components of the kits may be packaged either in aqueous media or in lyophilized form. When the components of the kit are provided in one and/or more liquid solutions, the liquid solution is a sterile aqueous solution.

Practice of the invention will be more fully understood from the foregoing examples, which are presented herein for illustrative purposes only, and should not be construed as limiting the invention in any way.

### EXAMPLES

### Example 1: Therapy of Tumor Targeted Superantigen and/or a Murine Anti-PD-1 Inhibitor in an MC38 Colon Cancer Mouse Model

This example describes a study testing the effect of a tumor targeted superantigen, C215Fab-SEA, and/or a murine anti-PD-1 antibody against a murine MC38-EpCAM colon tumor model in vivo. The therapy of tumor targeted superantigen and/or anti-PD-1 mAb was tested in a syngeneic tumor model using MC38 colon cancer cells transfected with the human colon carcinoma antigen EpCAM, which is recognized by the C215 antibody.

The tumor targeted superantigen C215Fab-SEA is a fusion protein which includes a tumor-reactive mAb (C215Fab) and the bacterial superantigen staphylococcal enterotoxin A (SEA). C215Fab-SEA was used as a model tumor targeted superantigen instead of, e.g., naptumomab estafenatox/ANYARA®, in order to facilitate in vivo murine experiments. Additionally, an anti-murine PD-1 antibody (RMP1-14, BIOXCELL) was used as a model anti-PD-1 antibody in order to facilitate in vivo murine experiments.

For the study, C57BL/6 mice were injected subcutaneously in the flank with 5×10⁵ MC38-EpCAM cells. Tumors were measured every 2-3 days (length×width) with a caliper. When tumor volume reached 30-60 mm³ (day 5), 10 mice in each treatment group were treated as follows: (i) daily IV injections of C215Fab-SEA (20 μg/mouse) on days 5-8, 12-15 and 19-22; and/or (ii) IP injections of anti-PD-1 mAb (50 μg/mouse) twice a week from day 8 to day 25. The control group was treated with PBS with the same mode of administration and regimen as the combination therapy group. Mice were sacrificed when tumors reached 2.25 cm³ or upon ulceration. During the study the animals were weighed at least twice weekly and were observed frequently for health and overt signs of any adverse treatment-related (TR) side effects.

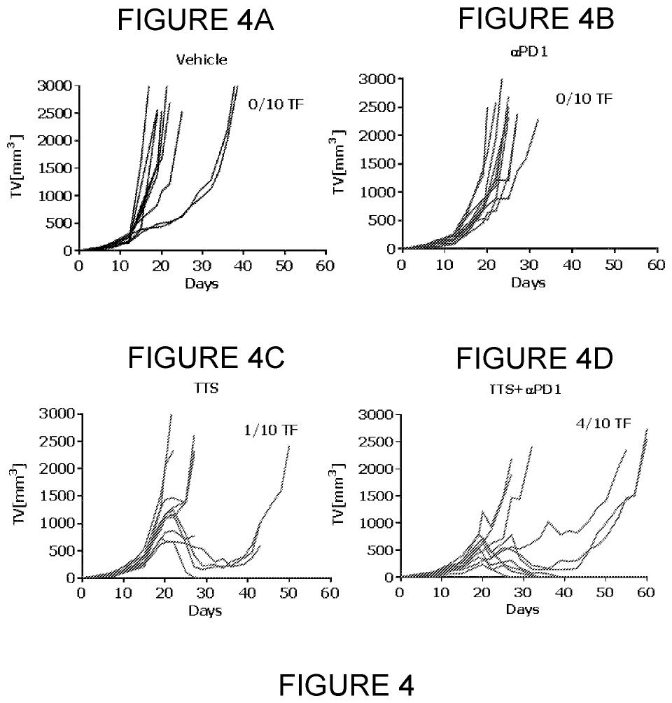

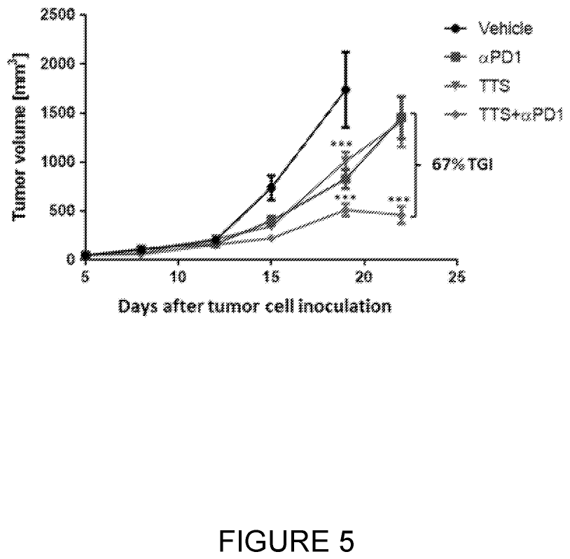

Tumor growth is shown FIGS. 4A-4D and FIG. 5. Slower tumor outgrowth was observed with monotherapy using either anti-PD-1 mAb or C215Fab-SEA (TTS) compared with the control. However, the combination of C215Fab-SEA with anti-PD-1 mAb had the most significant effect on tumor volume (TGI of 67%). In the C215Fab-SEA group, 1 of 10 mice experienced complete tumor regression (FIG. 4C). In contrast, treatment with C215Fab-SEA and anti-PD-1 mAb achieved complete tumor rejection in 4 of 10 mice (FIG. 4D).

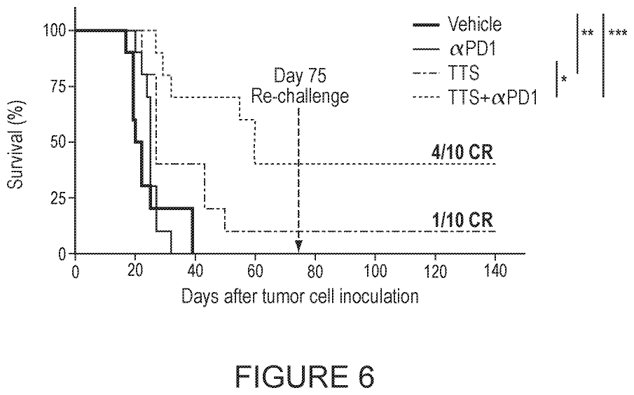

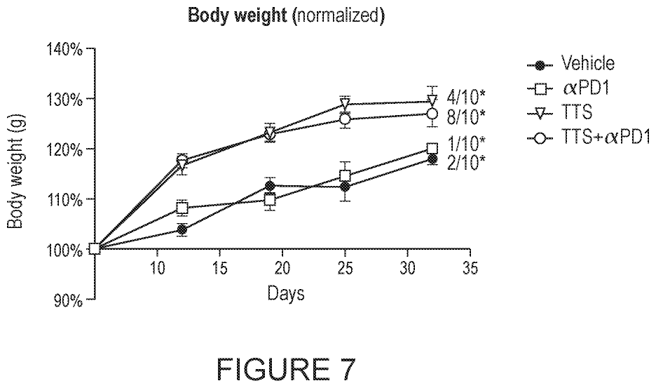

Survival is shown in FIG. 6. No survival benefit over the control was observed for the anti-PD-1 mAb treatment. Overall survival for the C215Fab-SEA treatment was significantly longer relative to control. The strongest effect was observed for the combination therapy, with significantly prolonged survival relative to each monotherapy. The combination treatment was well tolerated, and no signs of adverse events and/or body weight loss were recorded (FIG. 7).

These results demonstrate the potential of tumor targeted superantigen (e.g., C215Fab-SEA or naptumomab estafenatox/ANYARA®) optionally combined with an immunopotentiator (e.g., a PD-1 based inhibitor, e.g., an anti-PD-1 antibody) for the treatment of cancer, e.g., colon cancer. The therapy leads to potent induction of antitumor immunity and even complete response, i.e., tumor rejection, in certain subjects.

### Example 2: Immune Memory Following Therapy of Tumor Targeted Superantigen and/or a Murine Anti-PD-1 Inhibitor in an MC38 Colon Cancer Mouse Model

This example describes a study testing the long term effect of treatment of a MC38-EpCAM colon tumor model in vivo with a tumor targeted superantigen, C215Fab-SEA, and/or a murine anti-PD-1 antibody. In particular, cured mice from Example 1 were tested for immune memory by tumor re-challenge.

50 days following the last treatment with C215Fab-SEA and/or anti-PD-1 mAb described in Example 1, all surviving cured mice (complete responders, including 4 mice from the combination treatment group and 1 mouse from the C215Fab-SEA treatment group) were re-challenged with SC injections of 500K MC38-EpCAM tumor cells in the right flank and 500K MC38 parental tumor cells in the left flank. 5 naïve mice were used as controls.

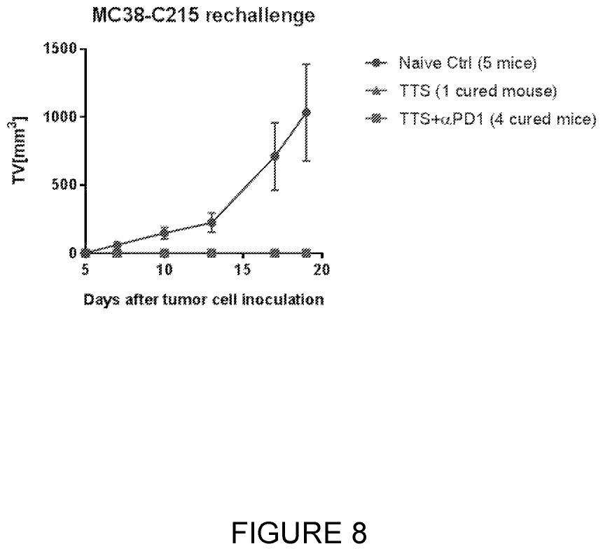

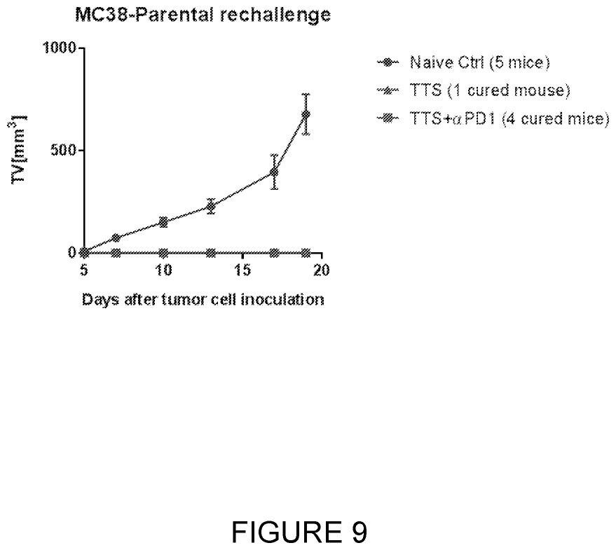

Tumor volume is shown in FIGS. 8 and 9. 100% of the naïve mice developed flank tumors in both sides. However, all the pre-treated mice completely rejected the second tumor challenge (in both sides). Notably, the pre-treated mice rejected the MC38 parental tumor cells even though they did not express the EpCAM cancer antigen against which the previous C215Fab-SEA treatment was targeted.

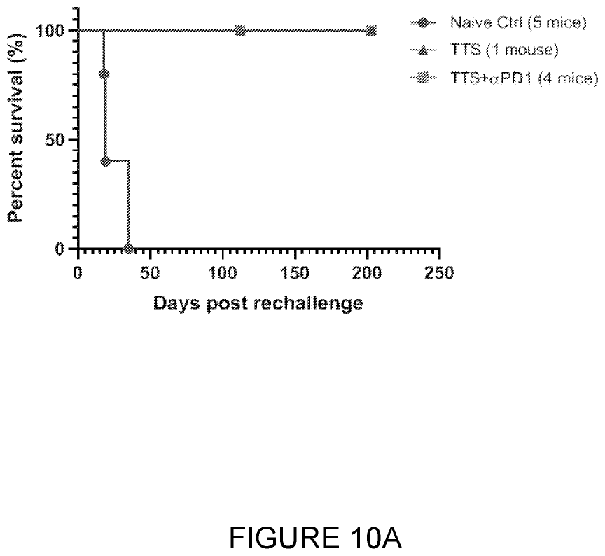

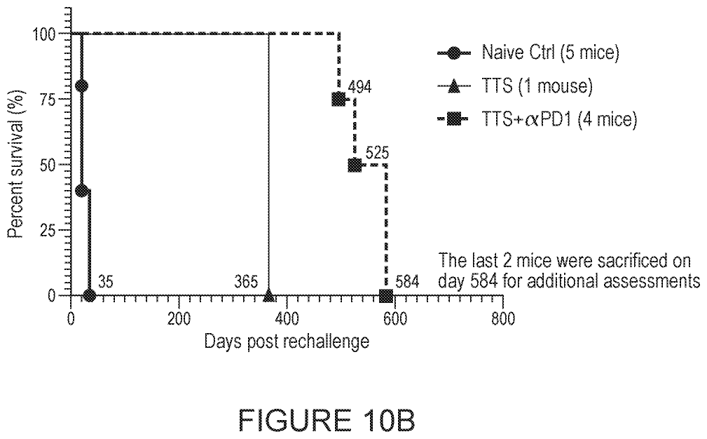

Initial survival results (up to 203 days post re-challenge) are shown in FIG. 10A, and complete survival results are shown in FIG. 10B. All the naïve mice died by day 35 of the study. The mouse previously treated with C215Fab-SEA alone died on day 365 of the study from age-related causes. Two of the mice previously treated with C215Fab-SEA in combination with anti-PD-1 mAb died on days 494 and 525 of the study from age-related causes (confirmed by necropsy). The remaining two mice previously treated with C215Fab-SEA in combination with anti-PD-1 mAb were sacrificed on day 584 of the study for additional assessments.

Together, these results demonstrate that the mice have long-term immunologic memory against the MC38-EpCAM tumor cells and the parental MC38 tumor cells. These results suggest that a therapy including tumor targeted superantigen (e.g., C215Fab-SEA or naptumomab estafenatox/ANYARA®) optionally together with an immunopotentiator (e.g., a PD-1 based inhibitor, e.g., an anti-PD-1 antibody) enhances tumor epitope spreading. In particular, these results suggest that a tumor targeted superantigen (e.g., C215Fab-SEA or naptumomab estafenatox/ANYARA®) enhances recognition of the targeted antigen (e.g., EpCAM or 5T4) by T cells, and, in addition, enhances recognition of other antigens by T cells, thereby inducing a long memory response against a tumor.

### Example 3: Immune Memory Following Therapy of Tumor Targeted Superantigen and/or a Murine Anti-PD-1 Inhibitor in an MC38 Colon Cancer Mouse Model

This example describes in vitro studies testing the long-term anti-cancer memory response of T cells in mice treated with tumor targeted superantigen and anti-PD-1 antibody.

On day 584 of the re-challenge experiment described in Example 2, the two surviving mice previously treated with C215Fab-SEA in combination with anti-PD-1 mAb were sacrificed. T cells were isolated from the sacrificed mice. T cells derived from naïve and MC38-EpCAM tumor bearing mice (TV=100 mm³; untreated) were used as controls. T cells were labeled with cell proliferation dye and cultured with either MC38 or MC38-EpCAM mouse colon tumor cells at 10:1 effector to target ratio for 5 days.

C215Fab-SEA can activate TRBV3 expressing T cells. In order to determine whether any long-term anti-cancer memory is mediated in part by T cells other than those directly activated by C215Fab-SEA, these studies only assayed the activity of TRBV3 negative CD4 and CD8 T cells.

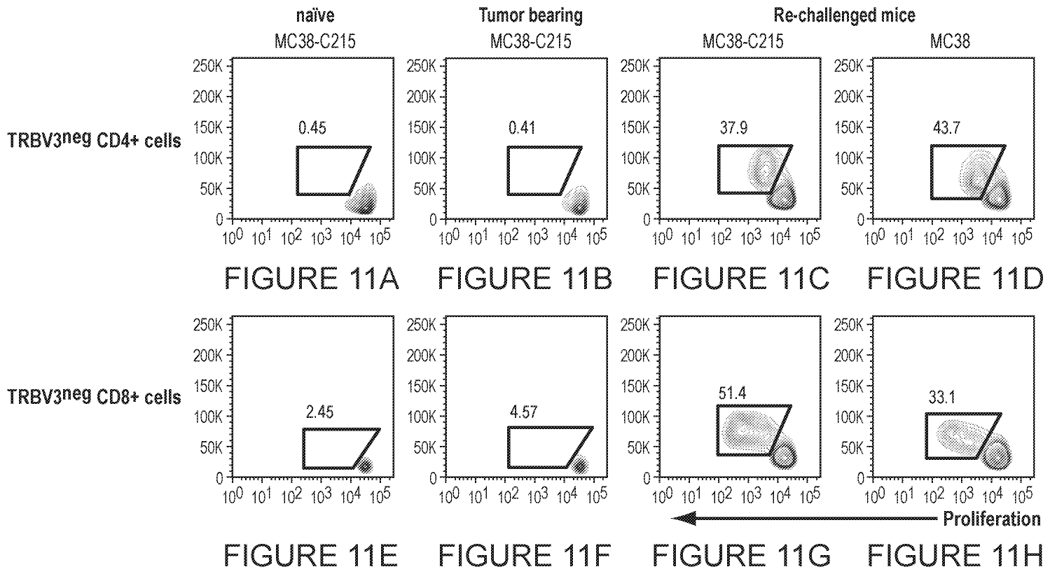

T cell proliferation in response to the tumor cells was analyzed by measuring the dilution of the proliferation dye by FACS. Results are shown in FIGS. 11A-11H. TRBV3 negative CD4 and CD8 T cells isolated from the control mice did not proliferate following incubation with the tumor cells (FIGS. 11A, 11B, 11E, and 11F). However, TRBV3 negative CD4 and CD8 T cells isolated from the re-challenged, cured mice significantly proliferated after incubation with both the MC38-EpCAM and MC38 cancer cells (FIGS. 11C, 11D, 11G, and 11H).

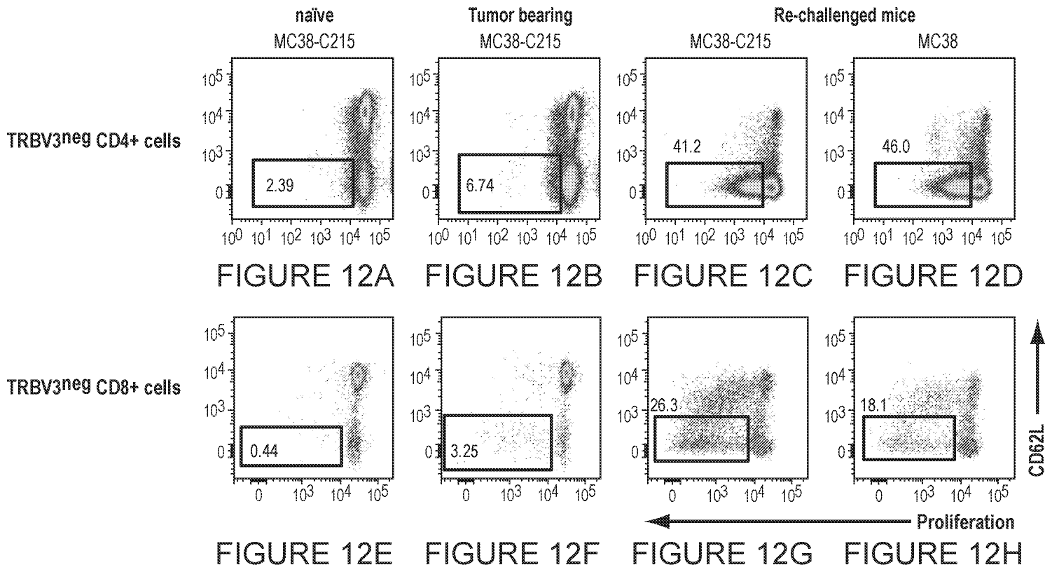

The differentiation of memory cells (CD44 high and CD62L positive) to activated effector cells (CD44 high and CD62L negative) was determined by the expression of CD62L and CD44 on the T cells. Results are shown in FIGS. 12A-12H. Consistent with earlier results, TRBV3 negative CD4 and CD8 T cells isolated from the control mice did not have a significant increase in the level of effector cells following incubation with the tumor cells (FIGS. 12A, 12B, 12E, and 12F). However, TRBV3 negative CD4 and CD8 T cells isolated from the re-challenged, cured mice had a significant increase in the level of effector cells after incubation with both the MC38-EpCAM and MC38 cancer cells (FIGS. 12C, 12D, 12G, and 12H).

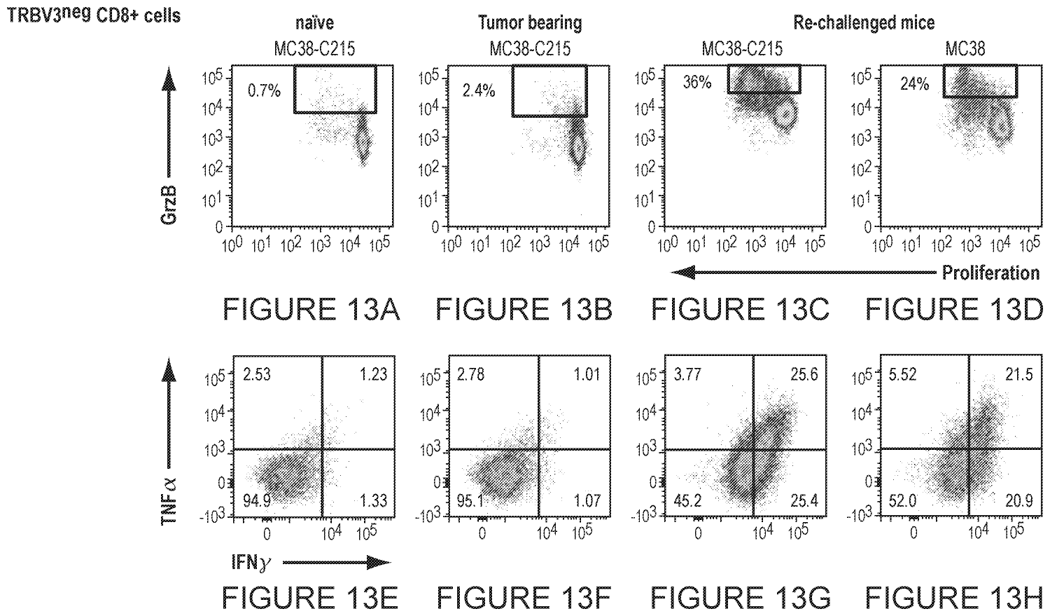

To assess the ability of the CD8 T cells to become cytotoxic T cells, T cells were restimulated with PMA and ionomycin and the levels of Granzyme B, TNFα and IFNγ were measured by FACS. Results are shown in FIGS. 13A-13H. Again, TRBV3 negative CD8 T cells isolated from the control mice had no detectable increase in Granzyme B, TNFα and IFNγ after restimulation (FIGS. 13A, 13B, 13E, and 13F). However, TRBV3 negative CD8 T cells isolated from the re-challenged, cured mice had a significant increase in the expression of Granzyme B and the secretion of IFNγ and TNFα after restimulation (FIGS. 13C, 13D, 13G, and 13H).

In summary, T cells isolated from mice treated with C215Fab-SEA in combination with anti-PD-1 mAb recognized tumor cells in vitro, even when the T cells were isolated from the mice 584 days after the rechallenge and 634 days after the last treatment. These results demonstrate that the mice treated with C215Fab-SEA in combination with anti-PD-1 mAb have long-term immunologic memory against the MC38 tumor cells. Additionally, this long-term immunologic memory response was not restricted to cells expressing the antigen targeted by C215Fab-SEA (EpCAM) or to T cells directly activated by C215Fab-SEA (TRBV3 expressing T cells). Accordingly, these results suggest that the long-term immunologic memory response following treatment with C215Fab-SEA in combination with anti-PD-1 mAb is induced by epitope spreading, and that treatment with C215Fab-SEA in combination with anti-PD-1 mAb generated an immune response to multiple tumor antigens.

### INCORPORATION BY REFERENCE

The entire disclosure of each of the patent and scientific documents referred to herein is incorporated by reference for all purposes.

### SOME NON-LIMITING EMBODIMENTS

Some non-limiting illustrative embodiments of this invention are listed below in the following numbered paragraphs:

1. A method of reducing the likelihood of a recurrence of a cancer in a subject in need thereof, the method comprising administering to the subject: (i) an effective amount of a superantigen conjugate comprising a superantigen covalently linked to a targeting moiety that binds a cancer antigen expressed by cancerous cells within the subject; and optionally (ii) an effective amount of an immunopotentiator.

2. A method of delaying a recurrence of a cancer in a subject in need thereof, the method comprising administering to the subject: (i) an effective amount of a superantigen conjugate comprising a superantigen covalently linked to a targeting moiety that binds a cancer antigen expressed by cancerous cells within the subject; and optionally (ii) an effective amount of an immunopotentiator.

3. A method of treating cancer and promoting anticancer immune memory and/or epitope spreading in a subject in need thereof, the method comprising administering to the subject: (i) an effective amount of a superantigen conjugate comprising a superantigen covalently linked to a targeting moiety that binds a cancer antigen expressed by cancerous cells within the subject; and optionally (ii) an effective amount of an immunopotentiator.

4. A method for inducing at least a first and second epitope-specific immune response in a subject with cancer, the method comprising administering to the subject: (i) an effective amount of a superantigen conjugate comprising a superantigen covalently linked to a targeting moiety that binds a cancer antigen expressed by cancerous cells within the subject; and optionally (ii) an effective amount of an immunopotentiator, wherein the first epitope-specific immune response is directed to the cancer antigen through the superantigen conjugate and the second epitope-specific immune response is not directed to the cancer antigen or the superantigen and is mediated by epitope spreading.

5. A method of mediating a long term (at least 6 months, 7 months, 8 month, 9 months, 10 months, 11 months, 1 year, 2 years or more) immune response against multiple, different cancer antigens expressed by a cancerous cell in a subject in need of treatment, the method comprising (or consisting essentially of) administering to the subject: (i) an effective amount of a superantigen conjugate comprising a superantigen covalently linked to a targeting moiety that binds a single type of cancer antigen expressed by cancerous cells within the subject; and optionally (ii) an effective amount of an immunopotentiator.

6. The method of any one of embodiments 1-5, wherein the cancer is a 5T4-expressing cancer.

7. The method of any one of embodiments 1-6, wherein the cancer is selected from breast cancer, bladder cancer, cervical cancer, colon cancer, colorectal cancer, endometrial cancer, gastric cancer, head and neck cancer, liver cancer, melanoma, mesothelioma, non-small cell lung cancer, ovarian cancer, pancreatic cancer, prostate cancer, renal cell cancer, and skin cancer.

8. The method of embodiment 7, wherein the cancer is colon or colorectal cancer.

9. A method of stimulating an immune response in a subject against a cancerous cell which does not express a cancer antigen, the method comprising administering to the subject: (i) an effective amount of a superantigen conjugate comprising a superantigen covalently linked to a targeting moiety that binds the cancer antigen; and optionally (ii) an effective amount of an immunopotentiator.

10. The method of embodiment 9, wherein the cancerous cell is selected from a breast cancer, bladder cancer, cervical cancer, colon cancer, colorectal cancer, endometrial cancer, gastric cancer, head and neck cancer, liver cancer, melanoma, mesothelioma, non-small cell lung cancer, ovarian cancer, pancreatic cancer, prostate cancer, renal cell cancer, and skin cancer cell.

11. The method of embodiment 10, wherein the cancerous cell is a colon or colorectal cancer cell.

12. A method of treating a colon or colorectal cancer in a subject in need thereof, the method comprising administering to the subject: (i) an effective amount of a superantigen conjugate comprising a superantigen covalently linked to a targeting moiety that binds a cancer antigen expressed by cancerous cells within the subject; and optionally (ii) an effective amount of an immunopotentiator.

13. The method of any one of embodiments 1-12, wherein the cancer antigen is selected from EpCAM and 5T4.

14. The method of embodiment 13, wherein the cancer antigen is 5T4.

15. The method of any one of embodiment 1-14, wherein the immunopotentiator is a PD-1 based inhibitor.

16. The method of embodiment 15, wherein the PD-1 based inhibitor is a PD-1 or PD-L1 inhibitor.

17. The method of any one of embodiment 1-16, wherein the subject has previously received a different anti-cancer therapy.

18. The method of embodiment 17, wherein the cancer is refractory to the anti-cancer therapy.

19. The method of embodiment 18, wherein the cancer recurred following the anti-cancer therapy

20. The method of any one of embodiments 17-19, wherein the anti-cancer therapy comprises a chimeric antigen receptor (CAR) T-cell or a bispecific T-cell engager (BiTE).

21. The method of any one of embodiments 1-20, wherein the superantigen conjugate is administered to the subject before, at the same time as, or after the PD-1 or PD-L1 inhibitor.

22. The method of any one of embodiments 1-21, wherein the superantigen comprises Staphylococcal enterotoxin A or an immunologically variant and/or fragment thereof.

23. The method of any one of embodiments 1-22, wherein the superantigen comprises the amino acid sequence of SEQ ID NO: 3, or an immunologically reactive variant and/or fragment thereof.

24. The method of any one of embodiments 1-23, wherein the targeting moiety is an antibody.

25. The method of embodiment 24, wherein the antibody is an anti-5T4 antibody.

26. The method of embodiment 25, wherein the anti-5T4 antibody comprises a Fab fragment that binds a 5T4 cancer antigen.

27. The method of embodiment 26, wherein the anti-5T4 antibody comprises a heavy chain comprising amino acid residues 1-222 of SEQ ID NO: 8 and a light chain comprising amino acid residues 1-214 of SEQ ID NO: 9.

28. The method of any one of embodiments 1-27, wherein the superantigen conjugate comprises a first protein chain comprising SEQ ID NO: 8 and a second protein chain comprising SEQ ID NO: 9.

29. The method of any one of embodiments 16-28, wherein the PD-1 inhibitor is an anti-PD-1 antibody.

30. The method of embodiment 29, wherein the anti-PD-1 antibody is selected from nivolumab pembrolizumab, and cemiplimab.

31. The method of any one of embodiment 16-30, wherein the PD-L1 inhibitor is an anti-PD-L1 antibody.

32. The method of embodiment 31, wherein the anti-PD-L1 antibody is selected from atezolizumab, avelumab, and durvalumab.

33. A superantigen covalently linked to a targeting moiety that binds a cancer antigen expressed by cancerous cells within the subject for (i) reducing the likelihood of a recurrence of a cancer in a subject, (ii) delaying reoccurrence of a cancer in a subject or (iii) promoting anticancer immune memory and/or epitope spreading in a subject.

34. A superantigen conjugate comprising a superantigen covalently linked to a targeting moiety that binds a cancer antigen expressed by cancerous cells within the subject for inducing at least a first and second epitope-specific immune response in a subject with cancer, wherein the first epitope-specific immune response is directed to the cancer antigen through the superantigen conjugate and the second epitope-specific immune response is not directed to the cancer antigen or the superantigen and is mediated by epitope spreading.

35. A superantigen conjugate comprising a superantigen covalently linked to a targeting moiety that binds a single type of cancer antigen expressed by cancerous cells within the subject for mediating a long term (at least 6 months, 7 months, 8 month, 9 months, 10 months, 11 months, 1 year, 2 years or more) immune response against multiple, different cancer antigens expressed by a cancerous cell in a subject in need of treatment.

36. The superantigen of any one of embodiments 33-35 for use in combination with an immunopotentiator.

37. The superantigen of any one of embodiments 33-36, wherein the cancer is a 5T4-expressing cancer.

38. The superantigen of any one of claims 33-37, wherein the cancer is selected from breast cancer, bladder cancer, cervical cancer, colon cancer, colorectal cancer, endometrial cancer, gastric cancer, head and neck cancer, liver cancer, melanoma, mesothelioma, non-small cell lung cancer, ovarian cancer, pancreatic cancer, prostate cancer, renal cell cancer, and skin cancer.

39. The superantigen of embodiment 39, wherein the cancer is colon or colorectal cancer.

40. A superantigen conjugate comprising a superantigen covalently linked to a targeting moiety that binds the cancer antigen for stimulating an immune response in a subject against a cancerous cell which does not express a cancer antigen.

41. The superantigen of embodiment 40, for use in combination with an immunopotentiator.

42. The superantigen of embodiment 41, wherein the cancerous cell is selected from a breast cancer, bladder cancer, cervical cancer, colon cancer, colorectal cancer, endometrial cancer, gastric cancer, head and neck cancer, liver cancer, melanoma, mesothelioma, non-small cell lung cancer, ovarian cancer, pancreatic cancer, prostate cancer, renal cell cancer, and skin cancer cell.

43. The superantigen of embodiment 42, wherein the cancerous cell is a colon or colorectal cancer cell.

44. The superantigen of any one of embodiments 33-43, wherein the cancer antigen is selected from EpCAM and 5T4.

45. The superantigen of embodiment 44, wherein the cancer antigen is 5T4.

46. The superantigen of any one of embodiments 33-45, wherein the immunopotentiator is a PD-1 based inhibitor.

47. The superantigen of embodiment 46, wherein the PD-1 based inhibitor is a PD-1 or PD-L1 inhibitor.

48. The superantigen of any one of embodiments 33-47, wherein the subject has previously received a different anti-cancer therapy.

49. The superantigen of embodiment 48, wherein the cancer is refractory to the anti-cancer therapy.

50. The superantigen of embodiment 49, wherein the cancer recurred following the anti-cancer therapy

51. The superantigen of any one of embodiments 48-50, wherein the anti-cancer therapy comprises a chimeric antigen receptor (CAR) T-cell or a bispecific T-cell engager (BiTE).

52. The superantigen of any one of embodiments 33-51, wherein the superantigen conjugate is administered to the subject before, at the same time as, or after the PD-1 or PD-L1 inhibitor.

53. The superantigen of any one of embodiments 33-52, wherein the superantigen comprises Staphylococcal enterotoxin A or an immunologically variant and/or fragment thereof.

54. The superantigen of any one of embodiments 33-53, wherein the superantigen comprises the amino acid sequence of SEQ ID NO: 3, or an immunologically reactive variant and/or fragment thereof.

55. The superantigen of any one of embodiments 33-54, wherein the targeting moiety is an antibody.

56. The superantigen of embodiment 55 wherein the antibody is an anti-5T4 antibody.

57. The method of embodiment 56, wherein the anti-5T4 antibody comprises a Fab fragment that binds a 5T4 cancer antigen.

58. The superantigen of embodiment 57, wherein the anti-5T4 antibody comprises a heavy chain comprising amino acid residues 1-222 of SEQ ID NO: 8 and a light chain comprising amino acid residues 1-214 of SEQ ID NO: 9.

59. The superantigen of any one of embodiments 33-58, wherein the superantigen conjugate comprises a first protein chain comprising SEQ ID NO: 8 and a second protein chain comprising SEQ ID NO: 9.

60. The superantigen of any one of embodiments 33-59, wherein the PD-1 inhibitor is an anti-PD-1 antibody.

61. The superantigen of embodiment 60, wherein the anti-PD-1 antibody is selected from nivolumab pembrolizumab, and cemiplimab.

62. The superantigen of any one of embodiments 33-61, wherein the PD-L1 inhibitor is an anti-PD-L1 antibody.

63. The superantigen of embodiment 62, wherein the anti-PD-L1 antibody is selected from atezolizumab, avelumab, and durvalumab.

64. A pharmaceutical composition for use in any one of the therapeutic indications defined by the methods of embodiments 1-32, comprising the superantigen of any one of embodiments 33-63.

65. The pharmaceutical composition of embodiment 64, indicated for use in combination with an immunostimulant.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A superantigen covalently linked to a targeting moiety that binds a cancer antigen expressed by cancerous cells within the subject for (i) reducing the likelihood of a recurrence of a cancer in a subject, (ii) delaying reoccurrence of a cancer in a subject or (iii) promoting anticancer immune memory and/or epitope spreading in a subject.

2. A superantigen conjugate comprising a superantigen covalently linked to a targeting moiety that binds a cancer antigen expressed by cancerous cells within the subject for inducing at least a first and second epitope-specific immune response in a subject with cancer, wherein the first epitope-specific immune response is directed to the cancer antigen through the superantigen conjugate and the second epitope-specific immune response is not directed to the cancer antigen or the superantigen and is mediated by epitope spreading.

3. A superantigen conjugate comprising a superantigen covalently linked to a targeting moiety that binds a single type of cancer antigen expressed by cancerous cells within the subject for mediating a long term (at least 6 months, 7 months, 8 month, 9 months, 10 months, 11 months, 1 year, 2 years or more) immune response against multiple, different cancer antigens expressed by a cancerous cell in a subject in need of treatment.

4. The superantigen of any one of claims 1-3 for use in combination with an immunopotentiator.

5. The superantigen of any one of claims 1-4, wherein the cancer is a 5T4-expressing cancer.

6. The superantigen of any one of claims 1-5, wherein the cancer is selected from breast cancer, bladder cancer, cervical cancer, colon cancer, colorectal cancer, endometrial cancer, gastric cancer, head and neck cancer, liver cancer, melanoma, mesothelioma, non-small cell lung cancer, ovarian cancer, pancreatic cancer, prostate cancer, renal cell cancer, and skin cancer.

7. The superantigen of claim 6, wherein the cancer is colon or colorectal cancer.

8. A superantigen conjugate comprising a superantigen covalently linked to a targeting moiety that binds the cancer antigen for stimulating an immune response in a subject against a cancerous cell which does not express a cancer antigen.

9. The superantigen of claim 8, for use in combination with an immunopotentiator.

10. The superantigen of claim 9, wherein the cancerous cell is selected from a breast cancer, bladder cancer, cervical cancer, colon cancer, colorectal cancer, endometrial cancer, gastric cancer, head and neck cancer, liver cancer, melanoma, mesothelioma, non-small cell lung cancer, ovarian cancer, pancreatic cancer, prostate cancer, renal cell cancer, and skin cancer cell.

11. The superantigen of claim 10, wherein the cancerous cell is a colon or colorectal cancer cell.

12. The superantigen of any one of claims 1-11, wherein the cancer antigen is selected from EpCAM and 5T4.

13. The superantigen of claim 12, wherein the cancer antigen is 5T4.

14. The superantigen of any one of claims 1-13, wherein the immunopotentiator is a PD-1 based inhibitor.

15. The superantigen of claim 14, wherein the PD-1 based inhibitor is a PD-1 or PD-L1 inhibitor.

16. The superantigen of any one of claims 1-15, wherein the subject has previously received a different anti-cancer therapy.

17. The superantigen of claim 16, wherein the cancer is refractory to the anti-cancer therapy.

18. The superantigen of claim 17, wherein the cancer recurred following the anti-cancer therapy

19. The superantigen of any one of claims 16-18, wherein the anti-cancer therapy comprises a chimeric antigen receptor (CAR) T-cell or a bispecific T-cell engager (BiTE).

20. The superantigen of any one of claims 1-19, wherein the superantigen conjugate is administered to the subject before, at the same time as, or after the PD-1 or PD-L1 inhibitor.

21. The superantigen of any one of claims 1-20, wherein the superantigen comprises Staphylococcal enterotoxin A or an immunologically variant and/or fragment thereof.

22. The superantigen of any one of claims 1-21, wherein the superantigen comprises the amino acid sequence of SEQ ID NO: 3, or an immunologically reactive variant and/or fragment thereof.

23. The superantigen of any one of claims 1-22, wherein the targeting moiety is an antibody.

24. The superantigen of claim 23 wherein the antibody is an anti-5T4 antibody.

25. The method of claim 24, wherein the anti-5T4 antibody comprises a Fab fragment that binds a 5T4 cancer antigen.

26. The superantigen of claim 25, wherein the anti-5T4 antibody comprises a heavy chain comprising amino acid residues 1-222 of SEQ ID NO: 8 and a light chain comprising amino acid residues 1-214 of SEQ ID NO: 9.

27. The superantigen of any one of claims 1-26, wherein the superantigen conjugate comprises a first protein chain comprising SEQ ID NO: 8 and a second protein chain comprising SEQ ID NO: 9.

28. The superantigen of any one of claims 15-27, wherein the PD-1 inhibitor is an anti-PD-1 antibody.

29. The superantigen of claim 28, wherein the anti-PD-1 antibody is selected from nivolumab pembrolizumab, and cemiplimab.

30. The superantigen of any one of claims 15-29, wherein the PD-L1 inhibitor is an anti-PD-L1 antibody.

31. The superantigen of claim 30, wherein the anti-PD-L1 antibody is selected from atezolizumab, avelumab, and durvalumab.
